(19) United States
(12) Patent Application Publication
(10) Pub. No.: US 2002/0143062 A1
(43) Pub. Date: Oct. 3, 2002

(54) Method to incorporate N-(4-hydroxyphenyl) retinamide in liposomes

(75) Inventors: Gabriel Lopez-Berestein, Bellaire, TX (US); Ana M. Tari, Houston, TX (US); Soo-Jeong Lim, Seoul (KR)

Correspondence Address: FULBRIGHT & JAWORSKI L.L.P., A REGISTERED LIMITED LIABILITY PARTNERSHIP, Suite 2400, 600 Congress Avenue, Austin, TX, 78701, US

(73) Assignee: Board of Regents, The University of Texas System

(21) Appl. No.: 09/982,113

(22) Filed: Oct. 17, 2001

Related U.S. Application Data
(60) Provisional application No. 60/241,445, filed on Oct. 17, 2000.

(51) Int. Cl. A61K 39/395; A61K 31/16; A61K 9/127

(52) U.S. Cl. 514/613; 424/155.1; 424/450

(57) ABSTRACT

Disclosed herein are simple and unique methods of methods of preparing liposomal compositions of N-(4-hydroxyphenyl) retinamide (4HPR) and/or other retinoids. Also disclosed are liposomal 4HPR compositions prepared by such methods, and use of such compositions in the treatment of diseases, such as breast cancer. This invention further provides methods for improving the efficacy of N-(4-hydroxyphenyl) retinamide (4HPR) as a chemopreventive agent in the presence of agents that potentiates its ability increase the expression of inducible nitric oxide synthase (iNOS, and NO production in cells.

### BACKGROUND OF THE INVENTION

This application claims benefit of the priority of U.S. Serial No. 60/241,445, filed Oct. 17, 2000, the entire contents of which is incorporated by reference.

1. Field of the Invention

The present invention relates generally to the fields of pharmaceutical retinoid compositions and methods of preparation thereof. More particularly, it concerns methods of preparing liposomal compositions of N-(4-hydroxyphenyl) retinamide (4HPR). The present invention further concerns such liposomal 4HPR compositions and methods of use in the treatment of diseases, such as cancer.

2. Description of Related Art

Retinoids are a large family of molecules encompassing thousands of members. Retinoic acid binds the retinoic acid receptor (RAR), fostering the formation of retinoid X receptor (RXR)/RAR hetrodimers, and subsequent induction of gene transcription (Chambon et al., 1991; Kliewer et al., 1992; Zhang et al., 1992). Some retinoids may directly effect second messengers (Evain-Brion et al., 1991).

Retinoids can play an important role in the development and differentiation of cells, and may have therapeutic efficacy in treating cancers and precancerous tumors (Kizaki et al., 1992). Acute promyelocytic leukemia has been treated with all-trans retinoic acid (RA) (Smith et al. 1992). Various amine, ester and amide derivatives of retinoic acid have been described as promoting cancer prophylaxis (Newton et al., 1980). For example, retinoic esters of L-cladinose exhibit anti-tumor activity (U.S. Pat. No. 5,096,713). retinic acid N-(carboxy)-phenylamindes and 7,8-dehydro-retinic acid N-(carboxy)-phenylamides describes these compounds as useful in preventing cancer and for therapy in bladder, mammary gland and skin cancer (Canadian Patent No. 1,127,170). In another instance, bifunctional retinoic acid esters have also been described as being used in cancer treatment and prevention (Shealy et al., 1988; U.S. Pat. No. 5,124,083). However, RA possesses toxic effects and retinoids are recognized as teratogens (Moon et al., 1992; Smith et al., 1992).

One retinoid with potent chemopreventative activity is N-(4-hydroxyphenyl) retinamide (4-HPR). 4-HPR has also demonstrated carcinogenesis for breast, bladder, lung, ovary and prostate cancer in animal models (Formelli et al., 1996; Pollard et al., 1991). Human trials using for chemopreventative effects in humans for prostate, cervix, skin, breast and lung have been conducted for 4-HPR (Fermelli et al., 1993; Kelloff, 1994). Additionally, various related derivatives also exhibit anti-neoplastic activity. For example, the retinamides R_{I} (4-(ethoxycarbylphenyl)-retinamide) and R_{II} (N-4(hydroxycarbophenyl) retinamide) have been described as cancer preventatives (Du et al., 1982; Song et al., 1984). In another instance, the use of N-(4-hydroxyphenyl)-all-trans-retinamide and N-(4-acyloxyphenyl)-all-trans-retinamide in breast cancer treatment and epithelial cancer prevention, respectively, has been described (U.S. Pat. Nos. 4,323,581 and 4,310,546).

As is the case with many retinoids, 4-HPR is poorly soluble in water, and various attempts have been made to create soluble compositions of 4-HPR and other retinoids. U.S. Pat. No. 4,665,098 describes a composition of 4-HPR that includes corn oil and a non-ionic surfactant to improve biodistribution. A composition of non-esterified fatty acids having 14-18 carbon atoms; monoglycerides which are monoesters of glycerol and fatty acids having 14-18 carbon atoms; lysophosphatidyl choline in which the fatty acid moiety has 14-18 carbon atoms and 4-HPR has also been described for oral administration, in U.S. Pat. No. 5,972,911. U.S. Pat. No. 5,534,261 describes preparation of various retinoids in lipid and liposome compositions for use in the prevention of post-operative cell adhesions. Liposome compositions containing 4-HPR and an antibody directed to disialoganglioside GD2 expressed on melanoma cells have been made and produced apoptosis in melanoma cells (Montaldo et al., 1999; Pagan et al., 1999).

Despite these advances, there is still a need for improved methods of liposome-retinoid production for therapeutic applications. Additionally, new compositions for storage or delivery of therapeutic retinoids would be a desirable improvement in the art. Additionally, the efficacy of liposomal retinoids may be enhanced by combining them with agents known to induce growth inhibition and apoptosis in cancer cells.

### SUMMARY OF THE INVENTION

The present invention overcomes deficiencies in the art by the disclosure of simplified methods for the production of liposome-retinoid compositions. Additionally, the present invention provides liposome-retinoid compositions with improved retinoid (e.g., N-(4-acyloxyphenyl)-all-trans-retinamide) incorporation efficiency. Also disclosed are methods and compositions for the treatment of diseases such as cancer.

The invention provides a method of preparing a pharmaceutical retinoid composition, comprising the step of admixing at least one retinoid or a derivative thereof with dimyristoyl phosphatidylcholine, soybean oil, tertiarybutyl alcohol and water, wherein the admixing produces a mixture comprising liposomes in combination with the at least one retinoid or derivative thereof.

In certain embodiments, the retinoid comprises N-(4-hydroxyphenyl) retinamide. In other embodiments, the derivative of a retinoid comprises at least one derivative of N-(4-hydroxyphenyl) retinamide.

The invention additionally provides a method of preparing a pharmaceutical retinoid composition, comprising the step of admixing N-(4-hydroxyphenyl) retinamide or a derivative thereof with dimyristoyl phosphatidylcholine, soybean oil, tertiarybutyl alcohol and water, wherein the admixing produces a mixture comprising liposomes in combination with the N-(4-hydroxyphenyl) retinamide or a derivative thereof.

The invention further provides a method of treating a subject having cancer, comprising administering to the individual a therapeutically effective amount of a composition comprising a retinoid, including but not limited to, N-(4-hydroxyphenyl) retamide, or a derivative thereof, encapsulated in a lipid material, wherein the lipid material comprises dimyristoyl phosphatidylcholine and soybean oil. In certain embodiments, the method further comprises administering at least one additional therapeutic agent to the individual.

The invention provides a pharmaceutical retinoid composition comprising N-(4-hydroxyphenyl) retinamide, or a derivative thereof, encapsulated in a lipid material, wherein the lipid material comprises dimyristoyl phosphatidylcholine and soybean oil.

In some embodiments, a method of the present invention further comprises the step of admixing at least one additional agent with one or more of the retinoid, dimyristoyl phosphatidylcholine, soybean oil, tertiarybutyl alcohol or water. In other embodiments, a composition of the present invention further comprises at least one additional agent. In certain aspects, the agent further comprises a linking moiety attached to the agent and one or more lipids attached to the linking moeity. In certain facets, the one or more lipids are the same as the at least one lipid. In other facets, the agent comprises a diagnostic agent. In some facets, the agent comprises a targeting agent. In specific attributes of the present invention, the targeting agent comprises at least one antibody that binds a tumor. In other aspects, the agent comprises an additional therapeutic agent. In some facets, the additional therapeutic agent comprises an anticancer agent. In specific attributes of the present invention, the anticancer agent is chemotherapy agent, a radiotherapy agent, an immune therapy agent, a genetic therapy agent, a hormonal therapy agent, a biological agent, an additional retinoid or a retinoid derivative.

In particular embodiments of the present invention, the dimyristoyl phosphatidylcholine and the soybean oil comprise a ratio of greater than or equal to 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2 or 99:1, and any range derivable therein, during admixing. In other embodiments of the present invention, the dimyristoyl phosphatidylcholine and the soybean oil comprise a ratio of greater than or equal to 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2 or 99:1, and any range derivable therein, in the liposome composition.

In some embodiments of the present invention, the ratio of retinoid or a derivative thereof to lipid is greater than or equal to 1:5, 1:6, 1:7, 1:8, 1:9, 1:10, 1:11, 1:12, 1:13, 1:14, 1:15, 1:16, 1:17, 1:18, 1:19 or 1:20, and any range derivable therein, during admixing. In some embodiments of the present invention, the ratio of retinoid or a derivative thereof to lipid is greater than or equal to 1:5, 1:6, 1:7, 1:8, 1:9, 1:10, 1:11, 1:12, 1:13, 1:14, 1:15, 1:16, 1:17, 1:18, 1:19 or 1:20, and any range derivable therein, in the liposome composition.

In additional embodiments, the water comprises greater than or equal to 1%, 2%, 3%, 4%, 5%, 6%, 7%, 8%, 9% or 10%, and any range derivable therein, during admixing.

In certain embodiments, the method further comprises freezing the mixture. In other embodiments, a compositition is frozen.

In other embodiments, the method further comprises lyophylizing the mixture. In some aspects, the lyophylizing produces a powder. In certain embodiments, the composition is comprised as a lyophylized material. In other aspects, the composition comprises a powder.

In some embodiments, the method further comprises resuspending the composition or mixture with an aqueous solvent. In certain aspects, the composition or mixture is resuspended with an aqueous solvent. In some aspects, the aqueous solvent comprises a pharmaceutically acceptable saline solution. In other embodiments, the composition is comprised in a pharmaceutically acceptable aqueous medium.

In additional embodiments, greater than or equal to 90%, 91%, 92%, 93%, 94%, 95%, 96%, 97%, 98%, 99%, and any range derivable therein, of the retinoid or a derivative thereof is incorporated into the liposomes.

Product composition comprising retinoids or derivatives thereof comprised in liposomes. Product composition comprising 4-HPR or derivatives thereof comprised in liposomes. Product composition comprising retinoid, or a derivative thereof, encapsulated in a lipid material, wherein the lipid material comprises dimyristoyl phosphatidylcholine and soybean oil. Product N-(4-hydroxyphenyl) retinamide, or a derivative thereof, encapsulated in a lipid material, wherein the lipid material comprises dimyristoyl phosphatidylcholine and soybean oil.

Product composition comprising retinoids or derivatives thereof comprised in liposomes obtainable by process comprising the step of admixing at least one retinoid or a derivative thereof with dimyristoyl phosphatidylcholine, soybean oil, tertiarybutyl alcohol and water, wherein the admixing produces a mixture comprising liposomes in combination with the at least one retinoid or derivative thereof Product composition comprising 4-HPR or derivatives thereof comprised in liposomes obtainable by process comprising the step of admixing at least one retinoid or a derivative thereof with dimyristoyl phosphatidylcholine, soybean oil, tertiarybutyl alcohol and water, wherein the admixing produces a mixture comprising liposomes in combination with the at least one 4-HPR or derivative thereof.

Product composition comprising retinoids or a derivative thereof comprised in liposomes for use as a medicament. Product composition comprising 4-HPR or a derivative thereof comprised in liposomes for use as a medicament.

Use of compound composition comprising retinoids or a derivative thereof comprised in liposomes for the manufacture or a medicament for the treatment of disease cancer. Use of compound composition comprising 4-HPR or a derivative thereof comprised in liposomes for the manufacture or a medicament for the treatment of disease cancer.

A method for manufacturing product liposome-retinoid comprising the steps of admixing at least one retinoid or a derivative thereof with dimyristoyl phosphatidylcholine, soybean oil, tertiarybutyl alcohol and water, wherein the admixing produces a mixture comprising liposomes in combination with the at least one retinoid or derivative thereof A method for manufacturing product liposome-4-HPR comprised in liposomes comprising the steps of admixing at least one 4-HPR or a derivative thereof with dimyristoyl phosphatidylcholine, soybean oil, tertiarybutyl alcohol and water, wherein the admixing produces a mixture comprising liposomes in combination with the at least one 4-HPR or derivative thereof.

In another embodiment, there is provided a method for increasing growth inhibitory effects of fenretinide on a cell comprising providing to a cell, in combination with fenretinide, one or more agents that increases the level of nitric oxide (NO) in the cell. The cell maybe a tumor cell such as a breast cancer cell which may further be comprised of an estrogen receptor (ER)-positive cell, or an estrogen receptor (ER)-negative cell. The fenretinide may be provided before the one or more agents, at the same time as the one or more agents, or after the one or more agents. Fenretinide may further be provided more than once, or daily for three months with monthly three-day interruptions. The other agent may be provided more than once.

The nucleic acid may be comprised of an expression construct encoding iNOS, interferon-γ or herceptin, a protein such as iNOS, interferon-γ or herceptin, or a chemopharmaceutical such as cyclosporin A.

In another embodiment, the tumor cell is a patient, or is part of a tumor mass in the patient. The method may provide for directly administrating to the tumor cell fenretinide or one or more agents. In yet another embodiment, the cell maybe provided with an additional anti-cancer therapy which may further employ radiation, a distinct chemotherapy, a distinct gene therapy, immunotherapy, or hormonal therapy. In yet another embodiment, fenretinide is provided in an amount sufficient to achieve an intracellular concentration of 0.1 μm, 0.5 μm, or 1.0 μm.

In a further embodiment, there is provided a method for treating cancer in a subject comprising providing to the subject, in combination, fenretinide and one or more agents that increases the level of nitric oxide (NO) in cancer cells in the subject. The cancer maybe breast cancer which may further be estrogen receptor (ER)-positive, or estrogen receptor (ER)-negative. The fenretinide may be provided at 10 mg/day, at 100 mg/day, or at 200 mg/day.

In still a further embodiment, there is provided a composition of matter comprising fenretinide and an agent that increases the level of nitric oxide (NO) in a cell given. The composition maybe encapsulated in a lipid material, for example, a liposome comprising dimyristoyl phosphatidylcholine and soybean oil.

Also envisioned as part of the invention is a kit comprising fenretinide and an agent that increases the level of nitric oxide (NO) in a cell.

In yet a further embodiment, there is provided a method for inhibiting metastasis in a subject having cancer comprising providing to the subject, in combination, fenretinide and one or more agents that increases the level of nitric oxide (NO) in cancer cells in the subject.

As used herein, “any integer derivable therein” means a integer between the numbers described in the specification, and “any range derivable therein” means any range selected from such numbers or integers.

As used herein the specification, “a” or “an” may mean one or more. As used herein in the claim(s), when used in conjunction with the word “comprising”, the words “a” or “an” may mean one or more than one. As used herein “another” may mean at least a second or more. As used herein, “any range derivable therein” means a range selected from the numbers described in the specification.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

Certain retinoids and derivatives thereof, particularly N-(4-hydroxyphenyl) retinamide, has shown the ability to induce apoptosis and inhibit cell growth in tumor cell types, such as breast cancer. However, because of their hydrophobicity and toxicity, improved methods for preparing retinoid compositions would be an advancement in the art.

The present invention overcomes the limitations of current methods of liposome-retinoid preparation by providing simplified methods of producing liposomes encapsulating retinoids. Additionally, novel liposome-retinoid compositions are disclosed with enhanced retinoid incorporation efficiency. It is contemplated that retinoid/lipid compositions may possess superior pharmaceutical properties, such as for example, an improved therapeutic index or biodistribution, than other retinoid/lipid formulations.

For example, an exemplary retinoid with desirable anti-cancer properties, N-(4-hydroxyphenyl) retinamide, was incorporated in liposomes prepared with various ratios of dimyristoyl-phosphatidylcholine (DMPC) and soybean oil. The incorporation efficiency of N-(4-hydroxyphenyl) retinamide increased up to 96% by optimizing the ratio of N-(4-hydroxyphenyl) retinamide to lipid mixture and the ratio of DMPC to soybean oil. This is a superior incorporation efficiency than described for other liposome preparation methods and compositions (Pagnan et al., 1998; Pagnan et al., 1999; Montaldo et al, 1999).

Additionally, preferred embodiments of the present invention comprise a simplified proticol for liposome-retinoid preparation, such as forgoing one or more steps of rotary evaporation using organic solvent, repeated cycles of freezethawing or active loading of 4-HPR into already formed liposomes. In certain specific embodiments, the present invention does not comprise rotary evaporation. In other specific embodiments the present invention does not comprise freeze-thawing. It is contemplated that methods incorporating one or more of these additional steps is less preferred, as these steps are less conducive to large scale production. Certain preferred embodiments of the methods disclosed here allows the separation procedure of unincorporated N-(4-hydroxyphenyl) retinamide from liposome-incorporated products to be made unnecessary, therefore, the methods of the present invention can be easier to do in large scale and to use in clinical settings, compared with other liposomal preparation method.

In certain specific embodiments, the present invention produces or comprises powdered forms of liposome-retinoid compositions. In specific aspects, powdered formulations are preferred. In certain facets, powdered formulations are more stable upon storage or otherwise possess a greater shelf-life than non-powdered formulations.

Further, the addition of one or more of these additional steps may produce compositions that can not be stored in powder formulation. Production of liposome-retinoid compositions in powdered compositions are more suitable for market due to improved shelf-life or stability in storage, and in certain embodiments the methods of the present invention may be used to produce such compositions.

The shelf stability of N-(4-hydroxyphenyl) retinamide at room temperature was significantly increased after incorporation in liposomes and liposomal N-(4-hydroxyphenyl) retinamide still maintained the growth-inhibitory activity of N-(4-hydroxyphenyl) retinamide itself The efficient incorporation of N-(4-hydroxyphenyl) retinamide by the method of the present invention may increase the in vitro and in vivo stability of N-(4-hydroxyphenyl) retinamide, compared with free N-(4-hydroxyphenyl) retinamide. It is contemplated that the methods of the present invention may also be applied in the preparation of liposomes comprising N-(4-hydroxyphenyl) retinamide derivatives, described herein, as well as be applied to other retinoids, particularly those with anti-tumor activity.

The method of the present invention may enhance the bioavailability of N-(4-hydroxyphenyl) retinamide, its derivatives, and/or other retinoids and agents at tumor sites by improving their pharmacokinetic properties. It is further contemplated that the biodistribution of lipid compositions of the present invention, particularly liposomes, may be altered by changing the size or lipid composition. In certain embodiments, it is contemplated that biodistribution may be altered by the conjugation of targeting ligands to the surface of the lipid composition surface, described herein. Such ligands may allow cell, tissue and/or organ specific accumulation of the drug.

In other embodiments, it is contemplated that improved toxicity profiles and/or therapeutic efficacy of a retinoid may be produced by combination with a lipid. Such aspects can be determined by procedures in cell cultures or experimental animals known to those of ordinary skill in the art or described herein. For example, one measure of drug effectiveness, cell toxicity, and safety of a compound is its therapeutic index: LD₅₀/ED₅₀. LD₅₀ is the median leathal dose, i.e., the dose lethal to 50% of the population, and ED₅₀ is the median effective dose, i.e., the dose required to achieve a specific effect in 50% of the population (e.g., anti-tumor activity). As would be understood by one of ordinary skill in the art, compositions having the highest therapeutic index (LD₅₀/ED₅₀) are desirable in clinical settings. In certain aspects, the therapeutic index should be greater than 2, preferably at least 10, more preferably at least 50. It is also contemplated that other pharmacokinetic or pharmacodynamic parameters (e.g., clearance, volume of distribution, half-life, drug release profiles) of the retinoid-lipid compositions of present invention may be superior to other preparations of retinoids. Such parameters are well known in the art (see, for example, Goodman and Gilman's “The Pharmacological Basis of Therapeutics”, pp. 18-32, 43-61, 66-78, Eighth Edition, 1990, incorporated herein by reference in relevant part).

This invention further comprises methods of increasing the efficacy of fenretinide as a chemopreventive agent in breast cancer by combining said retinoid with agents that increases its ability to increase the expression of inducible nitric oxide synthase (iNOS), and subsequently nitric oxide(NO) production, thereby inhibiting growth in cells. Agents which increase the ability of 4-HPR to induce iNOS expression and NO production include cytokines such as IFN-γ; immunosuppressive agents such as cyclosporin A, and agents which suppress or abrogate her2/neu oncogene expression such as herceptin

A. N-(4-Hydroxyphenyl) Retinamide

N-(4-hydroxyphenyl) retinamide, also known as 4-HPR or fenretinide, can induce growth inhibition and apoptosis in a variety of tumor cell types including breast cancer (U.S. Pat. Nos. 4,323,581 and 5,464,870, incorporated herein by reference). A synergistic effect against mammary cancer has been described for calcium glucarate and 4-HPR (see U.S. Pat. Nos. 5,008,291 and 5,010,107, incorporated herein by reference). Thus, in certain embodiments, it is contemplated that calcium glucarate may be incorporated into a liposome, and/or administered as an additional agent with the compositions of the present invention.

The 4-HPR incorporated in liposomes by the methods disclosed here can be used as a potent anticancer agent with improved properties. 4-HPR, and methods of making 4-HPR, have been described in U.S. Pat. Nos. 4,190,594, 4,323,581 and 5,399,757, each incorporated herein by reference.

1. Derivatives of N-(4-hydroxyphenyl) retinamide

In particular aspects, it is contemplated that derivatives of 4-HPR and related retinoids may be used instead of, or in combination with, 4-HPR in the compositions and methods of the present invention. As used herein, “retinoid” refers to 4-HPR, derivatives of 4-HPR, and other retinoids. As used herein, a “derivative of 4-HPR” refers to a compound whose chemical structure comprises 4-hydroxy moeity and a retinamide.

In some embodiments, derivatives of 4-HPR that may be used include, but are not limited to, C-glycoside and arylamide analogues of N-(4-hydroxyphenyl) retinamide-O-glucuronide, including but not limited to 4-(retinamido)phenyl-C-glucuronide, 4-(retinamido)phenyl-C-glucoside, 4-(retinamido)phenyl-C-xyloside, 4-(retinamido)benzyl-C-glucuronide, 4-(retinamido)benzyl-C-glucoside, 4-(retinamido)benzyl-C-xyloside; and retinoyl β-glucuronide analogues such as, for example, 1-(β-D-glucopyranosyl) retinamide and 1-(D-glucopyranosyluronosyl) retinamide, described in U.S. Pat. Nos. 5,516,792, 5,663,377, 5,599,953, 5,574,177, and Bhatnagar et al., 1991, each incorporated herein by reference.

U.S. Pat. No. 5,808,111, incorporated herein by reference, describes anticancer compounds with improved water solubility, including, for example, 1-(D-glucopyranosyl) acitretinamide; 1-(D-glucopyranuronosyl) acitretinamide; 1-(D-glycopyranuronosyl) acitretinamide; and metal salts thereof.

In other embodiments, retinoids that may be used in the present invention comprise those disclosed in U.S. Pat. No. 4,743,400, incorporated herein by reference. These retinoids include, for example, all-trans retinoyl chloride, all-trans-4-(methoxyphenyl) retinamide, 13-cis-4-(hydroxyphenyl) retinamide and all-trans-4-(ethoxyphenyl) retinamide.

U.S. Pat. No. 4,310,546, incorporated herein by reference, describes N-(4-acyloxyphenyl)-all-trans retinamides, such as, for example, N-(4-acetoxyphenyl)-all-trans-retinamide, N-(4-propionyloxyphenyl)-all-trans-retinamide and N-(4-n-butyryloxyphenyl)-all-trans-retinamide, all of which are contemplated for use in certain embodiments.

Another derivative of retinoic acid that may be used in certain embodiments is 4-acetamidophenyl retinoate (Tramposch et al., 1992). In other aspects, the retinoic acid derivatives 3,4-didehydroretinoic acid or CD367 (Torma et al., 1994) may be used. Retinoids such as N-(1H-tetrazol-5-yl)retinamide, N-ethylretinamide, fenretinide (N-(4-hydroxyphenyl) retinamide), 13-cis-N-ethylretinamide, N-butylretinamide, etretin (acitretin), etretinate, tretinoin (all-trans-retinoic acid) or isotretinoin (13-cis-retinoic acid) are contemplated for used in certain embodiments (Turton et al., 1992).

2. Retinoids

In other embodiments, one or more retinoids or retinoid derivatives, other than N-(4-hydroxyphenyl) retinamide and derivatives thereof, are contemplated for use in the present invention. Preferred retinoids and retinoid derivatives are those that possess a tissue or tumor specific targeting and/or therapeutic property (e.g., retinylacetate, retinylmethyl ether, 13-cis-retinoic acid). The retinoids and retinoid derivatives described herein are merely exemplary, and one of skill in the art will recognize that other retinoids and retinoid derivatives exist that may be combined in the methods and compositions of the present invention. All such retinoids and retinoid derivatives described herein or known to one of ordinary skill in the art are contemplated for used in the compositions and methods of the present invention. In addition, these retinoids and retinoid derivatives may be combined with other diagnostic and/or therapeutic agents and methods, described herein or that would be known to one of ordinary skill in the art.

U.S. Pat. No. 4,529,600, incorporated herein by reference, discloses chemopreventive agents for inhibiting tumor promotion in epithelial cells, specifically retinoic acid-4,4,18,18,18-d₅ and esters and amides thereof such as N-methyl retinamide-4,4,18,18,18-d₅, N-(2-hydroxyethyl) retinamide-4,4,18,18,18-d₅, N-isopropyl retinamide-4,4,18,18,18-d₅, N-butyl retinamide-4,4,18,18,18-d₅, N-hexyl retinamide-4,4,18,18,18-d₅, N-octyl retinamide-4,4,18,18,18-d₅, N,N-dimethyl retinamide-4,4,18,18,18-d₅, N-ethyl-N-methyl retinamide-4,4,18,18,18-d₅, N-isopropyl-N-methyl retinamide-4,4,18,18,18-d₅, N,N-diethyl retinamide-4,4,18,18,18-d₅, N-butyl-N-ethyl retinamide-4,4,18,18,18-d₅, N-methyl-N-octyl retinamide-4,4,18,18,18-d₅, N-phenyl retinamide-4,4,18,18,18-d₅, N-(4-hydroxyphenyl) retinamide-4,4,18,18,18-d₅, N-(4-methoxyphenyl) retinamide-4,4,18,18,18-d₅, N-naphthyl retinamide-4,4,18,18,18-d₅, N-methyl-N-phenyl retinamide-4,4,18,18,18-d₅, and N-tolyl retinamide-4,4,18,18,1-d₅, which are also contemplated for use in certain embodiments of the present invention.

U.S. Pat. No. 5,821,254, incorporated herein by reference, discloses 9-cis-retinoic acid and derivatives thereof, for use in treating cancers, particularly in combination with tomoxifen, raloxifene or derivatives thereof.

U.S. Pat. No. 5,124,083, incorporated herein by reference, discloses 3-substituted and 3,3-disubstituted all-trans-4-oxoretinoic acids and 3-substituted and 3,3-disubstituted 13-cis-4-oxoretinoic acids and their lower alkyl esters that possess cancer preventative and therapeutic properties. These derivatives include, for example, methyl 3-methyl-4-oxoretinoate, methyl 3-cinnamyl-4-oxoretinoate; methyl 3-ethyl-4-oxoretinoate; methyl 3-isopropyl-4-oxoretinoate; methyl 3-(tertiary-Butyl)-4-oxoretinoate; methyl 4-oxo-3-(2-propenyl)retinoate; methyl 4-oxo-3-(2-propynyl)retinoate; methyl 4-oxo-3-(phenylmethyl)retinoate; methyl 3-[(ethoxycarbonyl)methyl]-4-oxoretinoate; methyl 3,3-dimethyl-4-oxoretinoate; methyl 3.3-dipropynyl-4-oxoretinoate; methyl 3-cinnamyl-3-methyl-4-oxoretinoate; methyl 13-cis-4-oxoretinoate; methyl 13-cis-3-methyl-4-oxoretinoate; 3-substituted and 3,3-disubstituted 4-oxoretinoic acids; 3-methyl-4-oxoretinoic acid; 3-ethyl-4-oxoretionoic acid; 4-oxo-3-(2-propenyl)retinoic acid; 4-oxo-3-(2-propenyl)retinoic acid; 4-oxo-3-(2-propenyl)retinoic acid; 3-cinnamyl-4-oxoretinoic acid and 3.3-dimethyl-4-oxoretinoic acid.

Other retinoids with therapeutic activity on precancer or cancer cells sare described in U.S. Pat. No. 5,716,982, incorporated herein by reference, and include, for example, 3-acetyl-5-methyl-7-retinoyloxy-coumarin, 3-acetyl-7-retinoyloxy-8-methyl-coumarin, 3-acetyl-5-carboxy-7-retinoyloxy-coumarin, 3-acetyl 7-retinoyloxy-8-carboxy coumarin, 3-acetyl-6-chloro-7-retinoyloxy-coumarin, 3-acetyl-5-retinoyloxy-methylene-7-retinoyloxy-coumarin, 3-acetyl-5-retinoyloxy-6-carboxy-coumarin, 3-acetyl-7,8-diretinoyloxy-coumarin, 3-acetyl-5,7-diretinoyloxy-coumarin, 3-acetyl-5-ethoxycarbo-7-retinoyloxy-coumarin, 3-ethoxycarbo-5-dodecoxycarbo-7,8-retinoyloxy-coumarin, 3-acetyl-6-ethyl-7-retinoyloxy-coumarin, 3-acetyl-6-hexyl-7-retinoyloxy-coumarin, 3-ethoxycarbo-7-retinoyloxy-8-methyl-coumarin, 3,6-diacetyl-7-retinoyloxy-coumarin, 3-ethoxycarbo-7-retinoyloxy-coumarin, 3-ethoxycarbo-6-chloro-7-retinoyloxy-coumarin, 3-ethoxycarbo-5-carboxy-7-retinoyloxy-coumarin, 3-ethoxycarbo-7,8-diretinoyloxy-coumarin, 3-ethoxycarbo-5,7-diretinoyloxy-coumarin, 3-ethoxycarbo-6-ethyl-7-retinoyloxy-coumarin, 3-ethoxycarbo-7-retinoyloxy-8-acetyl-coumarin, 3-ethoxycarbo-5-retinoyloxy-6-benzoyl-coumarin, 3-carboxy-6-hexyl-7-retinoyloxy-coumarin, 3-carbamoyl-5-carboxy 7-retinoyloxy-coumarin, 3-carbamoyl-6-chloro-7-retinoyloxy-coumarin, 4-retinoyloxy-6-t-butyl-coumarin, 4-retinoyloxy-8-methyl-coumarin, 4,8-dimethyl-7-retinoyloxy-coumarin, 4-methyl-6-ethyl-7-retinoyloxy coumarin, 4-methyl-7-retinoyloxy-coumarin, 4-methyl-5,7-retinoyloxy-coumarin, 4-methyl-6-hexyl-7-retinoyloxy-coumarin, 4-retinoyloxy-6-methyl-coumarin, 4-methyl-7,8-diretinoyloxy-coumarin, 4-retinoyloxy-7-methyl-coumarin, 4-retinoyloxy-coumarin and 3-acetyl-7,8-diretinoyloxy-coumarin.

U.S. Pat. No. 4,565,863, incorporated herein by reference, describes retinoid carbohydrates with antineoplatic activity, such as, for example, D-glucopyranosyl-9-(4-methoxy-2,3,6-trimethylphenyl)-3,7-dimethyl-2,4,6,8-nonatetraenoate, D-ribofuranosyl 9-(4-methoxy-2,3,6-trimethylphenyl)-3,7-dimethyl-2,4,6,8-nonatetraenoate, 6,6-bis-O-[9-(4-methoxy-2,3,6,-trimethylphenyl)-3,7-dimethyl-2,4,6,8-nonat etraenoyl]-D-trehalose, [4-O-α-D-glucopyranosyl-D-glucopyranoxyl] 9-(4-methoxy-2,3,6-trimethylphenyl)-3,7-dimethyl-2,4,6,8-nonatetraenoate, [methyl] 2,6-bis-O-[9-(4-methoxy-2,3,6-trimethylphenyl)-3,7-dimethyl-2,4,6,8-nonate traenoyl]-α-D-glucopyranoside, [methyl]4O-[9-(4-methoxy-2,3,6-trimethylphenyl)-3,7-dimethyl-2,4,6,8-nonatetraenoyl]-α-D-glucopyranoside, [methyl] 2-O-[9-(4-methoxy-2,3,6-trimethylphenyl)-3,7-dimethyl-2,4,6,8-nonatetraenoyl]-α-D-glucopyranoside, [methyl] 6-O-[9-(4-methoxy-2,3,6-trimethylphenyl)-3,7-dimethyl-2,4,6,8-nonatetraenoyl]-α-D-glucopyranoside, 2-deoxy-2-[9-(4-methoxy-2,3,6-dimethylphenyl)-3,7-dimethyl-2,4,6,8-nonatetraenamido]-D-glucose, [2-[9-(4-methoxy-2,3,6-trimethylphenyl)-3,7-dimethyl-2,4,6,8-nonatetraenamido]ethyl]2-acetamido-3,4,6-tri-O-acetyl-2-deoxy-β-D-glucopyranoside, [2-[9-(4-methoxy-2,3,6-trimethylphenyl)-3,7-dimethyl-2,4,6,8-nonatetraenamido]ethyl] 2-acetamido-2-deoxy-β-D-glucopyranoside, 2-acetamido-2-deoxy-1,6-bis-O-[9(4-methoxy-2,3,6-trimethylphenyl)-3,7-dimethyl-2,4,6,8-nonatetraenoyl]-D-glucose, 1-O-[9-(4-methoxy-2,3,6-trimethylphenyl)-3,7-dimethyl-2,4,6,8-nonatetraenoyl]-L-rhamnose, 4,6,-ethylidene-1-O-[9-(4-methoxy-2,3,6-trimethylphenyl)-3,7-dimethyl-2,4,6,8-nonatetraenoyl]-D-glucose.

U.S. Pat. No. 4,863,969, incorporated herein by reference, describes 1,2,3,4-tetrahydro-1,1,4,4-tetramethyl-6-[(E)-alpha-methylstyryl]naphthalene, a retinoid derivative with anti-tumor properties. U.S. Pat. No. 5,096,924, incorporated herein by reference, discloses anti-tumor agent MI43-3711. U.S. Pat. No. 5,242,909, incorporated herein by reference, discloses 4-[2-[p-[(E)-2-(5,6,7,8-tetrahydro-5,5,8,8-tetramethyl-2-naphthyl)-propenyl]phenoxy]ethyl]-morpholine for us in the treatment of tumors, particularly in combination with cyclophosphamide.

U.S. Pat. No. 4,326,055, incorporated herein by reference, describes tumor inhibiting stillbene derivatives, such as, for example, α-methyl-p-[(E)-2-(5,6,7,8-tetrahydro-5,5,8,8-tetramethyl-2-naphthyl)propenyl]-benzyl alcohol; 1,2,3,4-tetrahydro-1,1,4,4-tetramethyl-6-[(E)-α-methyl-p-allylstryl]-naphthalene; 1,2,3,4-tetrahydro-1,1,4,4-tetramethyl-6-[(E)-α-methyl-p-vinylstyryl]naphthalene; 2-[p-[(E)-2-(5,6,7,8-tetrahydro-5,5,8,8-tetramethyl-2-naphthyl)propenyl]phenyl]-4,4-dimethyl-2-oxazoline; 2-hydroxyethyl p-[(E)-2-(5,6,7,8-tetrahydro-5,5,8,8-tetramethyl-2-naphthyl)-propenyl]-benzoate; 3-p-[[(E)-2-(5,6,7,8-tetrahydro-5,5,8,8-tetramethyl-2-naphthyl)propenyl]phenyl]-2-propen-1-yl acetate;4′-[(E)-2-(1,1,3,3-tetramethyl-5-indanyl)propenyl]-acetophenone; 4′-[(E)-2-(5,6,7,8-tetrahydro-5,5,8,8-tetramethyl-2-naphthyl)propenyl]-acetophenone; 4-nitrobenzyl p-[(E)-2-(5,6,7,8-tetrahydro-5,5,8,8-tetramethyl-2-naphthyl)propenyl]-benzoate; 6-[(E)-α,2,4-trimethylstyryl]-1,2,3,4-tetrahydro-1,1,4,4-tetramethyl-naphthalene; 6-[(E)-p,α-dimethylstyryl]-1,2,3,4-tetrahydro-1,1,4,4-tetramethyl-naphthalene; 6-[(E)-p-isopropyl-α-methylstyryl]-1,2,3,4-tetrahydro-1,1,4,4-tetramethylnaphthalene; D-[(E)-2-(5,6,7,8-tetrahydro-2-naphthyl)propenyl]-benzoic acid morpholide; p-[(E)-2-(1,1,3,3-tetramethyl-5-indanyl)-propenyl]-benzoic acid ethyl ester; p-[(E)-2-(3-Methoxy-5,6,7,8-tetrahydro-5,5,8,8-tetramethyl-2-naphthyl)propenyl]-benzoic acid ethyl ester; p-[(E)-2-(3-methoxy-5,6,7,8-tetrahydro-5,5,8,8-tetramethyl-2-naphthyl)-propenyl]-benzoic acid ethyl ester, p-[(E)-2-(5,6,7,8-tetrahydro-5,5,8,8-tetramethyl-2-naphthyl)propenyl]-benzyl methyl ether; p-[(E)-2-(5,6,7,8-tetrahydro-5,5,8,8-tetramethyl-2-naphthyl)-propenyl]-benzoic acid diethylamide; p-[(E)-2-(5,6,7,8-tetrahydro-5,5,8,8-tetramethyl-2-naphthyl)-propenyl]-benzoic acid monoethylamide; p-[(E)-2-(5,6,7,8-tetrahydro-5,5,8,8-tetramethyl-2-naphthyl)-propenyl]-benzoic acid; p-[(E)-2-(5,6,7,8-tetrahydro-5,5,8,8-tetramethyl-2-naphthyl)propenyl]-benzaldehyde; p-[(E)-2-(5,6,7,8-tetrahydro-5,5,8,8-tetramethyl-2-naphthyl)propenyl]-benzyl methyl ester; p-[(E)-2-(5,6,7,8-tetrahydro-5,5,8,8-tetramethyl-2-naphthyl)propenyl]-benzyl acetate; p-[(E)-2-(5,6,7,8-tetrahydro-5,5,8,8-tetramethyl-2-naphthyl)propenyl]-benzyl alcohol; p-[(E)-2-(5,6,7,8-tetrahydro-5,5,8,8-tetramethyl-2-naphthyl)propenyl]-2-methyl-benzoic acid ethyl ester; p-[(E)-2-(5,6,7,8-tetrahydro-5,5,8,8-tetramethyl-2-naphthyl)propenyl]-benzoic acid benzyl ester; p-[(E)-2(5,6,7,8-tetrahydro-5,5,8,8-tetramethyl-2-naphthyl)propenyl]benzoic acid ethyl ester and p-[(E)-2-(7-methoxy-1,1,3,3-tetramethyl-5-indanyl)propenyl]-benzoic acid ethyl ester;

U.S. Pat. Nos. 4,171,318, 4,231,944 and 4,395,575, each incorporated herein by reference, describe 9-substituted, 6- or 4-fluorine susbstituted retinoic acid anti-tumor derivatives, such as, for example, 2Z,4E-2-fluoro-3-methyl-5-(2,6-dichloro-3-methyl-4-methoxyphenyl)-pentadien-1-al, ethyl (E,Z,E,E)-9-(6-chloro-4-methoxy-2,3-dimethylphenyl)-4-fluoro-3,7-dimethyl-2,4,6,8-nonatetraenoate, methyl 2(E),4(E),6(Z),8(E)-3,7-dimethyl-6-fluoro-9-(2,3-dimethyl-4-methoxy-6-chlorophenyl)-nona-2,4,6,8-tetraenoate, ethyl (E,Z,E,E)-9-(3-chloro-2,4,6-trimethylphenyl)-4-fluoro-3,7-dimethyl-2,4,6,8-nonatetraenoate, methyl 2(E),4(E),6(Z),8(E),3,7-dimethyl-6-fluoro-9-(2,4,6-trimethyl-3-chlorophenyl)-nona-2,4,6,8-tetraenoate, ethyl (E,Z,E,E,)-9-(2-chloro-4-methoxy-3,6-dimethylphenyl)-4-fluoro-3,7-dimethyl-2,4,6,8-nonatetraenoate, methyl 2(E),4(E),6(Z),8(E)-3,7-dimethyl-6-fluoro-9-(2-chloro-3,6-dimethyl-4-methoxyphenyl)-nona-2,4,6,8-tetraenoate, ethyl (E,Z,E,E)-9-(2,3,6-trichloro-4-methoxyphenyl)-4-fluoro-3,7-dimethyl-2,4,6,8-nonatetraenoate, methyl 2(E),4(E),6(Z),8(E)-3,7-dimethyl-6-fluoro-9-(2,3,6-trichloro-4-methoxyphenyl)-nona-2,4,6,8-tetraenoate, ethyl (E,Z,E,E)-9-(2,6-dichloro-4-methoxy-3-methylphenyl)-4-fluoro-3,7-dimethyl-2,4,6,8-nonatetraenoate, methyl 2(E),4(E),6(Z),8(E)-3,7-dimethyl-6-fluoro-9-(2,6-dichloro-3-methyl-4-methoxyphenyl)-nona-2,4,6,8-tetraenoate, 2E,4E,6Z,8E-3,7-dimethyl-6-fluoro-9-(2,6-dichloro-3-methyl-4-methoxyphenyl)-2,4,6,8-nonatetraenoic acid, (2E,4E,6Z,8E)-3,7-dimethyl-6-fluoro-9-(2,6-dichloro-3-methyl-4-methoxyphenyl)-2,4,6,8-nonatetraenamide, N-ethyl (2E,4E,6Z,8E)-3,7-dimethyl-6-fluoro-9-(2,6-dichloro-3-methyl-4-methoxyphenyl)-2,4,6,8-nonatetraenamide, (2E,4E,6Z,8E)-3,7-dimethyl-6-fluoro-9-(2,6-dichloro-3-methyl-4-methoxyphenyl)-2,4,6,8-nonatetraen-1-ol, 2E,4E,6Z,8E-3,7-dimethyl-6-fluoro-9-(2,6-dichloro-3-methyl-4-methoxyphenyl)-2,4,6,8-nonatetraen-1-al, (2E,4E,6Z,8E)-3,7-dimethyl-6-fluoro-9-(2,6-dichloro-3-methyl-4-methoxyphenyl)-1-methoxy-2,4,6,8-nonatetraene and methyl 2(E),4(E),6(Z),8(E)-3,7-dimethyl-6-fluoro-9-(2,4,5-trimethyl-3-furyl)-nona -2,4,6,8-tetraenoate.

Some flavonoids and chalcones have been found to have anti-tumor properties, and are contemplated for use in the present invention. U.S. Pat. No. 4,960,908, incorporated herein by reference, describes isoflavone derivatives such as, for example, 5,7-diacetoxy-4′-methoxy-2-bromomethylisoflavone, 4′,5,7-triacetoxy-2-bromomethylisoflavone, 4′,5,7-trihydroxy-2-bromomethylisoflavone, 4′-ethoxalyloxy-5,7-dihydroxy-2-isoflavonecarboxylate, 2-(dimethylaminomethyl)-5,7-dihydroxy-4′-methoxyisoflavone hydrochloride, 4′,5,7,-trihydroxy-2-piperidinomethylisoflavone, 4′,5,7-trihydroxy-2-isoflavonecarboxamide, 4′,5,7-trihydroxy-N-methyl-2-isoflavonecarboxamide, 4′,5,7-trihydroxy-N-(2-hydroxyethyl)-2-isoflavonecarboxamide, 4′,5,7-trihydroxy-2-(methylthiomethyl)isoflavone, 4′5,7-trihydroxy-2-[2-thiazolin-2-ylthio)methyl]isolfavone, 3-[(4′,5,7-thrihydroxy-2-isoflavonyl)methyl]thio]-propionic acid, 4′,5,7-trihydroxy-2-isoflavonecarboxylate and isopropy 4′,5,7-trihydroxy-2-isoflavonecarboxylate.

U.S. Pat. No. 5,116,954, incorporated herein by reference, discloses various flavonoic compounds that possess anticancer properties, including, for example, oxo-1 phenyl-3-1H-naphto (2,1-b) pyran-5-acetonitrile; oxo-1-phenyl-3-(1H)-naphto (2.1-b) pyran-5-acetic Acid; oxo-4-phenyl-2-4H-naphto (2.3-b) pyran-1-acetic acid; oxo-4-phenyl-2-4H-naphto (1.2-b) pyran-10-acetic acid; methoxy-3-oxo-4-phenyl-2H-(1)-benzopyran-8-acetic acid; methoxy-5-oxo-4-phenyl-2H-4H-(1)-benzopyran-8-acetic acid; methoxy-2-phenyl)-2-oxo-4-4H-(1) benzopyran-8-acetic acid; hydroxy-3-oxo-4-phenyl-2-4H-(1)-benzopyran-8-acetic acid; hydroxy-5-oxo-4-phenyl-2-4H-(1)-benzopyran-8-acetic acid; hydroxy-7-oxo-4-phenyl-2-4H-(1)-benzopyran-8-acetic acid; hydroxy-2-phenyl)-2-oxo-4-4H-(1)-benzopyran-8-acetic acid; hydroxy-3-phenyl)-2-oxo-4-4H-(1)-benzopyran-8-acetic acid; hydroxy-4-phenyl)-2-oxo-4-4H-(1)-benzopyran-8-acetic acid; chlorohydrate of (diethylaminoethoxy-3-phenyl)-2-oxo-4-4H-benzopyran-8-acetic acid; phenoxy-2-phenyl-2-oxo-4-4H-(1)-benzopyran-8-acetic acid; fluoro-6-oxo-4-phenyl-2-4H-(1)-benzopyran-8-acetic acid; fluoro-2-phenyl)-2-oxo-4-4H-(1)-benzopyran-8-acetic acid; fluoro-4-phenyl)-2-oxo-4H-(1)-benzopyran-8-acetic acid; fluoro-3-phenyl)-2-oxo-4-4H-(1)-benzopyran-8-acetic acid; phenyl-4-phenyl)2-oxo-4-4H-(1)-benzopyran-8-acetic acid; (chloro-4-phenyl)-2-oxo-4-4H-(1)-benzopyran-8-acetic acid; (carboxy-4-phenyl)-2-oxo-4-4H-(1)-benzopyran-8-acetic acid; fluoro-2-phenyl)-4-phenyl)-2-oxo-4-4H-(1) benzopyran-8-acetic acid; (nitro-2-phenyl)-2-oxo-4-4H-(1)-benzopyran-8-acetic acid; (nitro-3-phenyl)-2-oxo-4-4H-(1)-benzopyran-8-acetic acid; (nitro-4-phenyl)-2-oxo-4-4H-(1)-benzopyran-8-acetic acid; (amino-3-phenyl)-2-oxo-4-4H-(1)-benzopyran-8-acetic acid; (amino-4-phenyl)-2-oxo-4-4H-(1)-benzopyran-8-acetic acid; oxo-4-phenyl-2-4H-(1)-benzopyran-5-acetic acid; oxo-4-phenyl-2-4H-(1)-benzopyran-7-acetic acid; trifluoromethyl-2-oxo-4-4H-(1)-benzopyran-8-acetic acid; oxo-4-phenyl-2-4H-(1)-benzothiopyran-8-acetic acid; oxo-4-phenyl-2-4H-(1)-benzothiopyran-8-acetic acid S,S-dioxide; oxo-4-phenyl-2-dihydro-1-4-quinoline-8-acetic acid; oxo-4-phenyl-2-4H (1)-benzoselenopyran-8-acetic acid; oxo-7-7H-benzo(c)xanthenyl-11-acetic acid; oxo-4-7-7H-dibenzo(c,h)xanthenyl-1-acetic acid; carboxymethyl-4-phenyl)-2-4H-(1) benzopyranone-4; carboxymethyl-3-phenyl)-2-4H-(1) benzopyranone-4; carboxymethyl-2-phenyl)-2-4H-(1) benzopyranone-4; ((oxo-4-phenyl-2-4H-(1) benzopyran-8-yl) methyl); ((oxo-4-phenyl-2-4H-(1) benzopyran-8-yl) methyl) phosphonic acid; (phenyl-2-oxo-4-4H-(1) benzopyran-8-yl)-2-acrylic acid; phenyl-3-(phenyl-2-oxo-4-4H-[1] benzopyran-8-yl)-2-acrylic acid; (bromo-2-phenyl)-3-(phenyl-2-oxo-4-4H-[1]benzopyran-8-yl)-3-acrylic acid; (pyridinyl-4)-3-(phenyl-2-oxo-4-4H-[1]benzopyran-8-yl)-3-acrylic acid; (pyridinyl-3)-3-(phenyl-2-oxo-4-4H-[1]benzopyran-3-acrylic acid; (chlorohydrate of [(methyl-4-piperazinyl) methyl]-8-phenyl-2-4H-[1]benzopyranone-4; (bromohydrate of N[imidazolinyl-2], N [(oxo-4-phenyl-2-4H-[1 l]benzopyran-8-yl) methyl]-dichloro-2-6-aniline; [(oxo-4-phenyl-2-4H-(1) benzopyran-8-yl methyl amino]-4-benzoic acid; N-[(oxo-4-phenyl-2-4H-(1) benzopyran-8-yl)methyl]N-methyl, amino-4-benzoic acid; [(oxo-4-phenyl-2-4H-[1]benzopyran-8-yl methylamino]-3, methyl-3, propanediol-1-3; chlorohydrate of (aminomethyl)-8-phenyl-2-4H-[1]benzopyranone-4; phenyl-2-(trimethoxy-3′,4,5-phenylaminomethyl)-8-4H-[1]benzopyranone-4; (acetyloxy-1-ethyl)-8-phenyl-2-4H-[1]benzopyranone-4; (hydroxy-1-ethyl-8-phenyl-2-4H-[1]benzopyranone-4; acetyl-8-phenyl-2-4H-1 benzopyranone-4; (bromoacetyl)-8-phenyl-2-4H-[1]benzopyranone-4; (amino 2-thiazol-4-yl)-8-phenyl-2-2H-[1]benzopyranone-4; [methyl-2-thiazol-4yl)-8-phenyl-2-4H-[1] benzopyranone-4; (imidazo [2,1-B]thiazol-6-yl)-8-phenyl-2-4H-[1]benzopyranone-4; (imidazo [1,2,-A]pyridin-2-yl)-8-phenyl-2-4H-[1]benzopyranone-4; (indolizin-2-yl)-8-phenyl-2-4H-[1]benzopyranone-4; phenyl-2-(phenyl-2-thiazol-4-yl)-8-4H-[1]benzopyranone-4; (dihydro-2-3-imidazo [2,1-B]thiazol-6-yl)-8-phenyl-2-4H-[1]benzopyranone-4; acetoxymethyl-10-phenyl-2-4H-naphto [1,2-b]pyranone-4; hydroxymethyl-10-phenyl-2-4H-naphto[1,2-b]pyranone-4; oxo-4-phenyl-2-4H-naphto[1,2-b]pyranone-4; oxo-4-phenyl-1-4H-[1]-benzopyran-8-acetate of (ethoxycarbonyl)-1-ethyl; hydroxy-4-methyl-5-(oxo-4-phenyl-2-4H-[1]-benzopyran-8-yl)-3-5H-furanone-2; (chloro-4-phenyl)-5-hydroxy-4-(oxo-4-phenyl-2-4H-[1]-benzopyran-8-yl]-3-5H-furanone-2; methyl-3-hydroxy-4-(oxo-4-phenyl-2-4H-[1]-benzopyran-8-yl)-5-5H-furanone-2; chlorhydrate of [(N,N-diethylamino)-2-ethoxy]-4-methyl-5-[oxo-4-phenyl-2-4H-[1]-benzopyran-8-yl]-3-5H-furanone-2; [dihydro-2-5-methy-5-oxo-2-(oxo-4-phenyl-2-4H-[1]-benzopyran-8-yl)-3-furan-4-yl]ethyl oxyacetate; dihydro-2-5-methyl-5-oxo-2-(oxo-4-phenyl-2-4H-[1]-benzopyran-8-yl)-3-furan-4-yl]oxyacetic acid; dimethyl carbamothioate of O-[dihydro-2,5-methyl-5-oxo-2-(oxo-4-phenyl-2-4H-[1]-benzopyran-8-yl)-3-fu rn-4-yl]; acetylthiomethyl-8-oxo-4-phenyl-2-4H-[1]-benzopyrane; mercaptomethyl-8-phenyl-2-4H-[1]-benzopyranone-4; (oxo-4-phenyl-2-4H-[1]-benzopyran-8-yl)-methyl]methyl thioacetate; (oxo-4-phenyl-2-4H-[1]-benzopyran-8-yl) methyl-thioacetic acid; oxalate of diethylamino-2-ethoxymethyl)-8-phenyl-2-4H-[1]-benzopyranone-4; [[hydroxy-2-(hydroxymethyl)-1-ethoxy]methyl]-8-phenyl-2-4H-[1]-benzopyranone-4; oxo-4-phenyl-2-4H-[1 ]-benzopyran-8-acetamide; oxo-4-phenyl-2-4H-[1]-benzopyran-8-thioacetamide; phenyl-2-[(phenyl-4-thiazol-2-yl)methyl]-8-4H-[1]-benzopyranone-4; [(oxo-4-phenyl-2-4H-[1]-benzopyran-8-yl) methyl]-2-thiazol-4-ethyl carboxylate; [oxo-4-phenyl-2-4H-{12]-benzopyran-8-yl) methyl-2-thiazol-4-carboxylic acid; [oxo-4-phenyl-2-4H-[1]-benzopyran-8-yl)methylene]-2-hydrazine carbothioamide; dihydro-4,5-[1H]-imidazol-2-yl-hydrazone bromhydrate of (oxo-4-phenyl-2-4H-[1]-benzopyran-8-yl) carboxaldehyde; (oxo-4-tetrahydro-2,3,5,6-4H-pyran-2-yl)-8-phenyl-2-4H-[1]-benzopyranone-4; (hydroxy-4-tetrahydro-3,4,5,6-2H-pyran-2-yl)-8-phenyl-2-4H-[1]-benzopyranone-4; oxo-4-4-(oxo-phenyl-2-4H-[1]-benzopyran-8-yl)-4-buten-2-oic acid; (oxo-4-phenyl-2-4H-[1]-benzopyran-8-yl)-2-hydroxy-2-acetic acid; (oxo-4-phenyl-2-4H-[1]-benzopyran-8-yl)-2-hydroxy-2-ethyl acetate; (oxo-4-phenyl-2-4H-[1]benzopyran-8-yl)-2-oxo-2-ethyl acetate; (oxo-4-phenyl-2-4H[1]benzopyran-8-yl)-2-oxo-2-acetate acid; methyl-2-(oxo-4-phenyl-2-4H-[1]benzopyran-8-Y)-2-methyl propanoate; methyl-2-(oxo-4-phenyl-2-4H-[1]benzopyran-8-yl)-2-propionic acid; oxo-4-phenyl-2-4H[1]-benzopyran-8-carboxaldehyde oxime; acetyl-8-phenyl-2-4H-[1]-benzopyranone-4-oxime; (morpholin-4-yl)-3-(oxo-4-phenyl-2-4H-[1]-Benzopyran-8-yl)-2-glutaronitrile; oxo-4-(oxo-4-phenyl-2-4H-[1]benzopyran-8-yl)-4-butyric acid; hydroxy-4-(oxo-4-phenyl-2°-4H-[1]benzopyran-8-y)-butyric acid; acetamido-2-ethoxycarbonyl-2-(oxo-4-phenyl-2-4H-[1 ]benzopyran-8-yl)-2 ethyl propionate; amino-2-(oxo-4-phenyl-2-4H-[1]benzopyran-8-yl)-3-propionic acid hydrochlorate; 2-(2-aminophenyl)-4-oxo-4H-[1]benzopyran-8-acetic acid; 2-(2-chlorophenyl)-4-oxo-4H-[1]benzopyran-8-acetic acid; 2-(3-chlorophenyl)-4-oxo-4H-[1]benzopyran-8-acetic acid; 2-(2-acetamidophenyl)-4-oxo-4H-[1]benzopyran-8-acetic acid; 2-(4-acetylphenyl)-4-oxo-4H-[1]benzopyran-8-acetic acid; 2-(4-acetamidophenyl)-4-oxo-4H-[1]benzopyran-8-acetic acid; 2-(2-diethylaminoethoxyphenyl)-4-oxo-4H-[1]-benzopyran-8-acetic acid; 2-(3-nitro-4-chlorophenyl)-4-oxo-4H-[1]-benzopyran-8-acetic acid; 2-(2,4-dimethoxyphenyl-4-oxo-4H-[1]-benzopyran-8-acetic acid; 2-(4-diethylaminoethoxyphenyl)-4-oxo-4H-[1]-benzopyran-8-acetic acid; 2-(4-carbamolyphenyl)-4-oxo-4H-[1]-benzopyran-8-acetic acid; [[methyl-2-thiazolyl-4-phenyl-2-oxo-4-4H[1]benzopyran-8-acetic acid; 2-(2-chlorophenyl)-4-oxo-4H-[1]-benzopyran-8-acetic acid; 2-(2-amino-4-thiazolylphenyl)-4-oxo-4H-[1]-benzopyran-8-acetic acid; 2-(3,5-dimethoxyphenyl)-4-oxo-4H-[1]-benzopyran-8-acetic acid; 2-(4-pyridyl)-4-oxo-4H-[1]-benzopyran-8-acetic acid; 2-(2-pyridyl)-4-oxo-4H-[1]-benzopyran-8-acetic acid; 2-(4-hexylphenyl)-4-oxo-4H-[1]-benzopyran-8-acetic acid; 2-(3-methylphenyl)-4-oxo-4H-[1]-benzopyran-8-acetic acid; 2-(4-benzoylphenyl)-4-oxo-4H-[1]-benzopyran-8-acetic acid; 2-(4-undecylphenyl)-4-oxo-4H-[1]-benzopyran-8-acetic acid; nitro-3-, phenyl-4-phenyl)-2-oxo-4H-[1]-benzopyran-8-acetic acid; 2-(4-trifluoromethylphenyl)-4-oxo-4H-[1]-benzopyran-8-acetic acid; 2-(4-dimethyltriazenylphenyl)-4-oxo-4H-[1]-benzopyran-8-acetic acid; 2-(3-nitro-4-methoxyphenyl)-4-oxo-4H-[1]-benzopyran-8-acetic acid and 2-(4-terbutylphenyl)-4-oxo-4H-[1]-benzopyran-8-acetic acid.

U.S. Pat. Nos. 5,703,130 and 5,968,940, each incorporated herein by reference, describe various cancer-inhibiting purine and chalcone retinoids, such as, for example, N-(3,5-di-tert-butyl-4-hydroxybenzoyl)-p-aminobenzoic acid p-hydroxy-anilide, N-(3,5-di-tert-butyl-4-hydroxybenzoyl)-p-aminobenzoic acid N-acetyl-p-aminophenol ester, N-(3,5-di-tert-butyl-4-hydroxybenzoyl)-p-aminobenzoic acid p-ethoxycarbonyl-anilide, N-(3,5-di-tert-butyl-4-hydroxybenzoyl)-p-aminobenzoic acid p-carboxy-anilide, 3′,5′-di-tert-butyl-4′-hydroxy-chalcone-4-carboxylic acid p-hydroxy-anilide, 3′,5′-di-tert-butyl-4′-hydroxy-chalcone-4-carboxylic acid N-acetyl-p-aminophenol ester, 3′,5′-di-tert-butyl-4′-hydroxy-chalcone-4-carboxylic acid p-ethoxycarbonyl-anilide, 3′,5′-di-tert-butyl-4′-hydroxy-chalcone-4-carboxylic acid p-carboxy-anilide, N-(3,5-di-tert-butyl-4-methoxybenzoyl)-p-aminobenzoic acid p-hydroxy-anilide, N-(3,5-di-tert-butyl-4-methoxybenzoyl)-p-aminobenzoic acid N-acetyl-p-aminophenol ester, N-(3,5-di-tert-butyl-4-methoxybenzoyl)-p-aminobenzoic acid p-ethoxycarbonyl-anilide, N-(3,5-di-tert-butyl-4-methoxybenzoyl)-p-aminobenzoic acid p-carboxy-anilide, 3′,5′-di-tert-butyl-4′-methoxy-chalcone-4-carboxylic acid p-hydroxy-anilide, 3′,5′-di-tert-butyl-4′-methoxy-chalcone-4-carboxylic acid N-acetyl-p-aminophenol ester, 3′,5′-di-tert-butyl-4′-methoxy-chalcone-4-carboxylic acid p-ethoxycarbonyl-anilide, 3′,5′-di-tert-butyl-4′-methoxy-chalcone-4-carboxylic acid p-carboxy-anilide, and isomers thereof.

U.S. Pat. Nos. 4,396,553, 4,870,219, 4,935,560, 4,990,703, 5,055,622, 5,001,276; and 5,030,764, each incorporated herein by reference, disclose styryl-tetrahydromaphthalene and indane derivatives for treating neoplasms. These patents specifically disclose such compounds as 1,2,3,4-tetrahydro-1,1,4,4,-tetramethyl-6-[(E)-α-methylstyryl]-2-naphthalenol; 1,2,3,4-tetrahydro-1,1,4,4,-tetramethyl-7-[(E)-α-methylstyryl]-2-naphthanenol, (E)-1,2,3,4-tetrahydro-5,8-dimethoxy-6(α-methylstyryl) napthalene, 5-(p-hydroxy-cc-methylstyryl)-1,1,3,3,-tetramethyl-2-indanone, 5-(p-hydroxy-α-methylstyryl-1,1,3,3-tetramethyl-2-indanol, 1,2,3,4-tetrahydro-1,1,4,4,7-pentamethyl-6-[(E)-α-methylstyryl]naphthalene, 1,2,3,4-tetrahydro-1,1,4,4-tetramethyl-6-(α-methylstyryl)-7-octylnaphthalene, (E)-1,2,3,4-tetrahydro-7-methoxy-6-(α-methylstyryl)-1,1,4,4-tetramethylnaphthalene, (E)-6-chloro-1,2,3,4-tetrahydro-1,1,4,4-tetramethyl-7-(α-methylstyry-1)naphthalene, 6-(α-ethylstyryl)-1,2,3,4-tetrahydro-1,1,4,4-tetramethylnaphthalene, (E)-1,2,3,4-tetrahydro-1,1,4,4-tetramethyl-6-styryl-naphthalene, 5-(p-fluoro-α-methylstyryl)-1,1,3,3-tetramethylindane, 5-(p-chloro-α-methylstyryl)-1,1,3,3-tetramethylindane, 5-(p-iodo-α-methylstyryl)-1,1,3,3-tetramethylindane, (E)-6-(p-fluoro-α-methylstyryl)-1,1,3,4-tetrahydro-1,1,4,4-tetramethylnaphthalene, 6-1,2,3,4-tetrahydro-1,1,4,4-tetramethylnaphthalene, (E)-6-[p-nitro-α-methylstyryl]-1,2,3,4-tetrahydro-1,1,4,4-tetramethylnaphthalene, p-[2-(5,6,7,8-tetrahydro-5,5,8,8-tetramethyl-2-naphthyl)propenyl]aniline, N,N-diethyl-p-[(E)-2-(5,6,7,8-tetrahydro-5,5,8,8-tetramethyl-2-naphthyl)propenyl]aniline, methyl p-[2-(5,6,7,8-tetrahydro-5,5,8,8-tetramethyl-2-naphthyl)propenyl]phenylsulphide, ethyl p-[2-(5,6,7,8-tetrahydro-5,5,8,8-tetramethyl-2-naphthyl)propenyl]phenylsulphide, methyl p-[2-(5,6,7,8-tetrahydro-5,5,8,8-tetramethyl-2-naphthyl)propenyl]phenylsulphoxide, ethyl p-[2-(5,6,7,8-tetrahydro-5,5,8,8-tetramethyl-2-naphthyl)propenyl]phenylsuphhoxide, N-ethyl-p-[2-(5,6,7,8-tetrahydro-5,5,8,8-tetramethyl-2-naphthyl)propenyl]benzenesulphonamide, ethyl p-[2-(5,6,7,8-tetrahydro-5,5,8,8-tetramethyl-2-naphthyl)propenyl]benzenesulphinate, ethyl p-[2-(5,6,7,8-tetrahydro-5,5,8,8-tetramethyl-2-naphthyl)propenyl]phenylsulphone, methyl p-[2-(5,6,7,8-tetrahydro-5,5,8,8-tetramethyl-2-naphthyl)propenyl]phenylsulphone, allyl p-[2-(5,6,7,8-tetrahydro-5,5,8,8-tetramethyl-2-naphthyl)propenyl]-phenylsulphone, 2,2,2-trifluoroethyl p-[2-(5,6,7,8-tetrahydro-5,5,8,8-tetramethyl-2-naphthyl)propenyl]phenylsulphone, propyl p-[2-(5,6,7,8-tetrahydro-5,5,8,8-tetramethyl-2-naphthyl)propenyl]phenylsulphone, isopropyl p-[2-(5,6,7,8-tetrahydro-5,5,8,8-tetramethyl-2-naphthyl)propenyl]phenylsulphone, 1-methylpropyl p-[2-(5,6,7,8-tetrahydro-5,5,8,8-tetramethyl-2-naphthyl)propenyl]phenylsulphone, 2-[[p-[2-(5,6,7,8-tetrahydro-5,5,8,8-tetramethyl-2-naphthyl)propenyl]phenyl]sulphonyl]-ethanol, vinyl p-[2-(5,6,7,8-tetrahydro-5,5,8,8-tetramethyl-2-naphthyl)propenyl]phenylsulphone, 2-chloroethyl p-[2-(5,6,7,8-tetrahydro-5,5,8,8-tetramethyl-2-naphthyl)propenyl]phenylsulphone, ethyl[[p-[2-(5,6,7,8-tetrahydro-5,5,8,8-tetramethyl-2-naphthyl)propenyl]phenyl]sulphonyl]acetate, p-[2-(5,6,7,8-tetrahydro-5,5,8,8-tetramethyl-2-naphthyl)propenyl]benzenesulphonic acid, sodium p-[2-(5,6,7,8-tetrahydro-5,5,8,8-tetramethyl-2-naphthyl)propenyl]benzenesulphinate and sodium p-[2-(5,6,7,8-tetrahydro-5,5,8,8-tetramethyl-2-naphthyl)propenyl]benzenesulphonate.

U.S. Pat. Nos. 4,602,034 and 4,783,533, each incorporated herein by reference, describe various anti-tumor (oxo-4-4H-(1)-benzopyran-8-yl) alkanoic acid related compounds, including, for example, (cyclohexyl-2-oxo-4-4H-[1]-benzopyran-8-yl)acetic acid; (diethylamino-2-ethyl)[oxo-4-phenyl-2-4H-[1]-benzopyran-8-yl-]acetate; (dimethylamino-3-propyle) [oxo-4-phenyl-2-4H-[1]-benzopyran-8-yl]acetate; (diphenyl-2,3-oxo-4-4H-[1]-benzopyran-8-yl)acetic acid; (diphenyl-2,3-oxo-4-4H-[1]-benzopyran-8-yl)acetonitrile; (methoxy-6-oxo-4-phenyl-2-4H-[1]-benzopyran-8-yl)acetate of (N,N-diethylamino)-2-ethyl; (methoxy-6-oxo-4-phenyl-2-4H-[1]-benzopyran-8-yl)acetic acid; (methoxy-6-oxo-4-phenyl-2-4H-[1]-benzopyran-8-yl)acetonitrile; (methyl-2-oxo-4-4H-[1]-benzopyran-8-yl)acetic acid; (methyl-2-oxo-4-4H-[1]-benzopyran-8yl)acetonitrile; (morpholinyl-4)-2-ethyl) [oxo-4-phenyl-2-4H-[1]-benzopyran-8-yl]acetate; (oxo-4-phenyl-3-4H-[1]-benzopyran-8-yl)acetic acid; [(dimethoxy-3,4-phenyl)-2-oxo-4-4H-[1]-benzopyran-8-yl]acetic acid; [(dimethoxy-3,4-phenyl)-2-oxo-4-4H-[1]-benzopyran-8-yl]acetonitrile; [(furyl-2)-2-oxo-4-4H-[1]-benzopyran-8-yl]acetic acid; [(furyl-2)-2-oxo-4-4H-[1]-benzopyran-8-yl] acetonitrile; [(methoxy-3-phenyl)-2-oxo-4-4H-[1]-benzopyran-8-yl]acetic acid; [(methoxy-3-phenyl)-2-oxo-4-4H-[1]-benzopyran-8-yl]acetonitrile; [(methoxy-4-phenyl)-2-oxo-4-4H-[1]-benzopyran-8-yl]acetate of (N,N-diethylamino)-2-ethyl; [(methoxy-4-phenyl)-2-oxo-4-4H-[1]-benzopyran-8-yl]acetic acid, [(methoxy-4-phenyl)-2-oxo-4-4H-[1]-benzopyran-8-yl]actonitrile; [(methyl-4-phenyl-)-2-oxo-4-4H-[1]-benzopyran-8-yl]acetic acid; [(naphtyl-2)-2-oxo-4-4H-[1]-benzopyran-8-yl]acetic acid; [(naphtyl-2-)-2-oxo-4-4H-[1]-benzopyran-8-yl]acetonitrile; [cyclohexyl-2-oxo-4-4H-[1]-benzopyran-8-yl]acetonitrile; [methyl-6-oxo-4-phenyl-2-4H-[1]-benzopyran-8-yl]acetic acid; [methyl-6-oxo-4-phenyl-2-4H-[1]-benzopyran-8-yl]acetonitrile; [oxo-4-(phenylmethyl)-2-4H-[1]-benzopyran-8-yl]acetic acid; [oxo-4-(phenylmethyl)-2-4H-[1]-benzopyran-8-yl]acetonitrile; [oxo-4-(thenyl-2)-2-4H-[1]-benzopyran-8-yl]acetate; [oxo-4-(thenyl-2)-2-4H-[1]-benzopyran-8-yl]acetonitrile; [oxo-4-(thenyl-2)-4H-[1]-benzopyran-8-yl]acetate of (N-N diethylamino)-2-ethyl; [oxo-4-phenyl-2-4H-[1]-benzopyran-8-yl]-2 propionic acid; [oxo-4-phenyl-2-4H-[1]-benzopyran-8-yl]-2-propionate or (N,N-diethylamino)-2-ethyl; [oxo-4-phenyl-2-4H-[1]-benzopyran-8-yl]-3-propionic acid; [oxo-4-phenyl-2-4H-[1]-benzopyran-8-yl]acetic acid; [oxo-4-phenyl-2-4H[1]-benzopyran-8-yl]acetonitrile; diethyl[(oxo-4-phenyl-2-4H-[1]-benzopyran-8-yl)methyl]-2-propanediate; diethyl[methyl-6-oxo-4-phenyl-2-4H-[1]-benzopyran-8-yl]-2-methyl-2, malonate; diethyl[oxo-4-phenyl-2-4H-[1]-benzopyran-8-yl]-2, methyl-2-melonate; diethylamino-2-ethyl[oxo-4-phenyl-2-4H-[1]-benzopyran-8-yl]-3-propenoate; diethylamino-2-ethyl[oxo-4-phenyl-2-4H-[1]-benzopyran-8-yl]acetate; ethyl [oxo-4-phenyl-2-4H-[1]-bezopyran-8-yl-] acetate; hydroxy-2-ethyl[oxo-4-phenyl-2-4H-[1]-benzopyran-8-yl]acetate; methyl[methyl-6-oxo-4-phenyl-2-4H-[1]-benzopyran-8-yl]acetate; methyl-6-oxo-4-phenyl-2-4H-[1]-benzopyran-8-yl]-2, propionic acid; N-[N′N′-diethylamino-2-ethyl] [oxo-4-phenyl-2-4H-[1]-benzopyran-8-yl]acetamide; oxo-4-phenyl-2-4H-[1]-benzopyran-8-yl]-2-propionate of (N,N-diethylamino)-2-ethyl; oxo-4-phenyl-2-4H-[1]-benzopyran-8-yl]-3-propenoic acid; oxo-4-phenyl-2-4H-[1]-benzopyran-8-yl]carboxaldehyde and (oxo-4-phenyl-3-4H-[1]-benzopyran-8-yl)acetic acid.

Other representative retinoids and retinoid derivatives contemplated for use in the invention are disclosed in EP 0 100 839 B1; EP 0 11 325 B1; EP 0 263 492 A1; EP 0 263 493 A2; EP 0 274 104 A2; EP 0 303 915 B1; EP 0 552 624 A1; EP 0439 042 A1; EP 0568 898 A1; EP-A-010,208; EP-A-010,209; EP-A-033,095; EP-A-09,776; Canadian Patent No. 1,127,170; French Patent 2,293,193; U.K. patent application GB 2 190 378 A; U.S. Pat. Nos. 4,108,880; 4,126,693; 4,126,698; 4,171,318; 4,190,594; 4,200,647; 4,231,944; 4,310,546; 4,323,581; 4,326,055; 4,395,575; 4,396,553; 4,523,042; 4,529,600; 4,565,863; 4,602,034; 4,713,465; 4,743,400; 4,783,533; 4,863,969; 4,870,219; 4,883,613; 4,894,480; 4,900,478; 4,935,560; 4,960,908; 4,990,703; 5,001,276; 5,030,764; 5,030,767; 5,055,622; 5,075,333; 5,096,713; 5,096,924; 5,116,954; 5,124,083; 5,158,773; 5,192,544; 5,242,909; 5,399,757; 5,516,792; 5,574,177; 5,599,953; 5,663,377; 5,703,130; 5,716,982; 5,968,940; Blazsek et al., 1991; Cassady et al., 1991; Castaigne et al., 1990; Chomienne et al., 1990; Dawson et al., 1981; Dawson, 1984; Degos, 1992; Doepner et al., 1992; Du et al., 1982; Edwards et al., 1990; Han et al., 1990; Harvey et al., 1988; Harvey et al., 1990; Ito et al., 1991; Jing et al., 1992; Kagechika et al., 1989; Kistler et al., 1990; Kizaki et al., 1992; Lo Coco et al., 1991; M. Boehm et al., 1994; Middleton et al., 1992; Nair et al., 1991; Newton et al., 1980; Oikawa et al., 1993; Shealy et al., 1988; Skrede et al., 1991; Smith et al., 1992; Song et al., 1984; Torma et al., 1994; Tramposch et al., 1992; Turton et al., 1992; Wilihite et al., 1992; and the Annals of the New York Academy of Sciences, Vol. 359, each incorporated herein by reference.

B. Lipid Compositions

In certain embodiments, the present invention concerns a novel composition comprising one or more lipids associated with at least one retinoid, such as, for example, N-(4-hydroxyphenyl) retinamide or a derivative thereof. A lipid is a substance that is characteristically insoluble in water and extractable with an organic solvent. Compounds than those specifically described herein are understood by one of skill in the art as lipids, and are encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof.

1. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

2. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

3. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

4. Lipid Composition Structures

A retinoid, such as, for example, N-(4-hydroxyphenyl) retinamide or a derivative thereof, associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid/retinoid associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine(Gibco BRL)-retinoid or Superfect (Qiagen)-retinoid complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10%to about 35% phosphatidyl choline, and about 1% of a drug. Thus, it is contemplated that lipid compositions of the present invention may comprise any of the lipids, lipid types or other components in any combination or percentage range.

a. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modern Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

b. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

5. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a lipid and/or retinoid may be, for example, encapsulated in the aqueous interior of a liposome, interspersed within the lipid bilayer of a liposome, attached to a liposome via a linking molecule that is associated with both the liposome and the retinoid, entrapped in a liposome, complexed with a liposome, etc.

a. Making Liposomes

In a preferred embodiment, the compositions of the present invention are made using very simple steps. Example 1 describes the simplified procedure, wherein a retinoid (e.g., 4-HPR) was mixed with lipid mixture comprising dimyristoyl-phosphatidylcholine (DMPC) and soybean oil in tertiarybutyl alcohol-water mixture. It is contemplated that in the present invention, such reagents may be admixed using standard liquid mixing protocols and apparatus, as would be known to those of skill in the art.

Additionally, a liposome used according to the present invention can be made by different methods, as would be known to one of ordinary skill in the art.

For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the retinoid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the retinoid or other component is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster, 1983, Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000× g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiements, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al., 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/retinoid or liposomes only of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. Liposomal encapsulation has resulted in a lower toxicity and a longer serum half-life for such compounds (Gabizon et al., 1990).

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

b. Liposome Targeting

Association of a retinoid with a liposome may improve biodistribution and other properties of the retinoid. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposome/retinoid composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of a retinoid. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

i. Cross-linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. Table 1 details certain hetero-bifunctional cross-linkers considered useful in the present invention.

TABLE 1
HETERO-BIFUNCTIONAL CROSS-LINKERS

| | | | Spacer Arm |
|---|---|---|---|
| | | | Length\after |
| Linker | Reactive Toward | Advantages and Applications | cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

In instances where a particular polypeptide does not contain a residue amenable for a given cross-linking reagent in its native sequence, conservative genetic or synthetic amino acid changes in the primary sequence can be utilized.

ii. Targeting Ligands

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., Chem. Phys. Lipids 40:347 (1986)) In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., Chem. Phys. Lipids 40:347 (1986)) For example, disialoganglioside GD2 is a tumor antigen that has been identified neuroectodermal origin tumors, such as neuroblastoma, melanoma, small-cell lung carcenoma, glioma and certain sarcomas (Mujoo et al., 1986, Schulz et al., 1984). Liposomes containing anti-disialoganglioside GD2 monoclonal antibodies have been used to aid the targeting of the liposomes to cells expressing the tumor antigen (Montaldo et al., 1999; Pagan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

Still further, a retinoid may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific retinoid delivery and/or targeting vehicle may comprise a specific binding ligand in combination with a liposome. The retinoid to be delivered are housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In certain embodiments, a receptor-mediated delivery and/or targeting vehicles comprise a cell receptor-specific ligand and a retinoid-binding agent. Others comprise a cell receptor-specific ligand to which retinoid to be delivered has been operatively attached. For example, several ligands have been used for receptor-mediated gene transfer (Wu and Wu, 1987; Wagner et al., 1990; Perales et al., 1994; Myers, EPO 0273085), which establishes the operability of the technique. In another example, specific delivery in the context of another mammalian cell type has been described (Wu and Wu, 1993; incorporated herein by reference).

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

C. Liposome/Nucleic Acid Combinations

It is contemplated that when the liposome/retinoid composition comprises a cell or tissue specific nucleic acid, this technique may have applicability in the present invention. In certain embodiments, lipid-based non-viral formulations provide an alternative to viral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

An exemplary method for targeting viral particles to cells that lack a single cell-specific marker has been described (U.S. Pat. No. 5,849,718). In this method, for example, antibody A may have specificity for tumor, but also for normal heart and lung tissue, while antibody B has specificity for tumor but also normal liver cells. The use of antibody A or antibody B alone to deliver an anti-proliferative nucleic acid to the tumor would possibly result in unwanted damage to heart and lung or liver cells. However, antibody A and antibody B can be used together for improved cell targeting. Thus, antibody A is coupled to a gene encoding an anti-proliferative nucleic acid and is delivered, via a receptor mediated uptake system, to tumor as well as heart and lung tissue. However, the gene is not transcribed in these cells as they lack a necessary transcription factor. Antibody B is coupled to a universally active gene encoding the transcription factor necessary for the transcription of the anti-proliferative nucleic acid and is delivered to tumor and liver cells. Therefore, in heart and lung cells only the inactive anti-proliferative nucleic acid is delivered, where it is not transcribed, leading to no adverse effects. In liver cells, the gene encoding the transcription factor is delivered and transcribed, but has no effect because no an anti-proliferative nucleic acid gene is present. In tumor cells, however, both genes are delivered and the transcription factor can activate transcription of the anti-proliferative nucleic acid, leading to tumor-specific toxic effects.

The addition of targeting ligands for gene delivery for the treatment of hyperproliferative diseases permits the delivery of genes whose gene products are more toxic than do non-targeted systems. Examples of the more toxic genes that can be delivered includes pro-apoptotic genes such as Bax and Bak plus genes derived from viruses and other pathogens such as the adenoviral E4orf4 and the E. coli purine nucleoside phosphorylase, a so-called “suicide gene” which converts the prodrug 6-methylpurine deoxyriboside to toxic purine 6-methylpurine. Other examples of suicide genes used with prodrug therapy are the E. coli cytosine deaminase gene and the HSV thymidine kinase gene.

It is also possible to utilize untargeted or targeted lipid complexes to generate recombinant or modified viruses in vivo. For example, two or more plasmids could be used to introduce retroviral sequences plus a therapeutic gene into a hyperproliferative cell. Retroviral proteins provided in trans from one of the plasmids would permit packaging of the second, therapeutic gene-carrying plasmid. Transduced cells, therefore, would become a site for production of non-replicative retroviruses carrying the therapeutic gene. These retroviruses would then be capable of infecting nearby cells. The promoter for the therapeutic gene may or may not be inducible or tissue specific.

Similarly, the transferred nucleic acid may represent the DNA for a replication competent or conditionally replicating viral genome, such as an adenoviral genome that lacks all or part of the adenoviral E1a or E2b region or that has one or more tissue-specific or inducible promoters driving transcription from the E1a and/or E1b regions. This replicating or conditional replicating nucleic acid may or may not contain an additional therapeutic gene such as a tumor suppressor gene or anti-oncogene.

d. Lipid Administration

The actual dosage amount of a lipid composition (e.g., a liposome-retinoid) administered to a patient can be determined by physical and physiological factors such as body weight, severity of condition, idiopathy of the patient and on the route of administration. With these considerations in mind, the dosage of a lipid composition for a particular subject and/or course of treatment can readily be determined.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally and/or using aerosol, injection, infusion, continuous infusion, localized perfusion bathing target cells directly or via a catheter and/or lavage.

C. Cancer Treatments

In order to increase the effectiveness of a lipid/retinoid composition, it may be desirable to combine these compositions of the with an agent effective in the treatment of hyperproliferative disease, such as, for example, an anti-cancer agent. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

More generally, such an agent would be provided in a combined amount with an lipid/retinoid composition effective to kill or inhibit proliferation of a cancer cell. This process may involve contacting the cell(s) with an agent(s) and the lipid/retinoid at the same time. This may be achieved by contacting the cell, tissue or organism with a single composition or pharmacological formulation that includes both a lipid/retinoid and one or more agents, or by contacting the cell with two or more distinct compositions or formulations, at the same time, wherein one composition includes a lipid and a retinoid and the other includes one or more agents.

The terms “contacted” and “exposed,” when applied to a cell, tissue or organism, are used herein to describe the process by which a therapeutic construct of the invention and/or another agent, such as for example a chemotherapeutic or radiotherapeutic agent, are delivered to a target cell, tissue or organism or are placed in direct juxtaposition with the target cell, tissue or organism. To achieve cell killing or stasis, the lipid/retinoid composition and/or additional agent(s) are delivered to one or more cells in a combined amount effective to kill the cell(s) or prevent them from dividing.

The lipid/retinoid compostion may precede, be co-current with and/or follow the other agent(s) by intervals ranging from minutes to weeks. In embodiments where the lipid/retionoid composition, and other agent(s) are applied separately to a cell, tissue or organism, one would generally ensure that a significant period of time did not expire between the time of each delivery, such that the lipid/retinoid composition and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) as the lipid/retinoid composition. In other aspects, one or more agents may be administered within of from about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, to about 48 hours or more prior to and/or after administering the lipid/retinoid compostion. In certain other embodiments, an agent may be administered within of from about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20, to about 21 days prior to and/or after administering the lipid/retinoid compostion. In some situations, it may be desirable to extend the time period for treatment significantly, however, where several weeks (e.g., about 1, about 2, about 3, about 4, about 5, about 6, about 7 or about 8 weeks or more) lapse between the respective administrations.

Various combination regimens of the lipid/retinoid composition and one or more agents may be employed. Non-limiting examples of such combinations are shown below, wherein a composition of the invention is “A” and an agent is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the composition of the present invention to a cell, tissue or organism may follow general protocols for the administration of chemotherapeutics, taking into account the toxicity, if any. It is expected that the treatment cycles would be repeated as necessary. In particular embodiments, it is contemplated that various additional agents may be applied in any combination with the present invention.

1. Chemotherapeutic Agents

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, anti-tumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat, for example, chronic leukemia, non-Hodgkin's lymphoma, Hodgkin's disease, multiple myeloma, and particular cancers of the breast, lung, and ovary. An alkylating agent, may include, but is not limited to, a nitrogen mustard, an ethylenimene, a methylmelamine, an alkyl sulfonate, a nitrosourea or a triazines.

They include but are not limited to: busulfan, chlorambucil, cisplatin, cyclophosphamide (cytoxan), dacarbazine, ifosfamide, mechlorethamine (mustargen), and melphalan. In specific aspects, troglitazaone can be used to treat cancer in combination with any one or more of these alkylating agents, some of which are discussed below.

i. Nitrogen Mustards

A nitrogen mustard may be, but is not limited to, mechlorethamine (HN₂), which is used for Hodgkin's disease and non-Hodgkin's lymphomas; cyclophosphamide and/or ifosfamide, which are used in treating such cancers as acute or chronic lymphocytic leukemias, Hodgkin's disease, non-Hodgkin's lymphomas, multiple myeloma, neuroblastoma, breast, ovary, lung, Wilm's tumor, cervix testis and soft tissue sarcomas; melphalan (L-sarcolysin), which has been used to treat such cancers as multiple myeloma, breast and ovary; and chlorambucil, which has been used to treat diseases such as, for example, chronic lymphatic (lymphocytic) leukemia, malignant lymphomas including lymphosarcoma, giant follicular lymphoma, Hodgkin's disease and non-Hodgkin's lymphomas.

a. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. For example, after a single oral doses of about 0.6 mg/kg to about 1.2 mg/kg, peak plasma chlorambucil levels are reached within one hour and the terminal half-life of the parent drug is estimated at about 1.5 hours. About 0.1 mg/kg/day to about 0.2 mg/kg/day or about 36 mg/m²/day to about 6 mg/m²/day or alternatively about 0.4 mg/kg may be used for antineoplastic treatment. Chlorambucil is not curative by itself but may produce clinically useful palliation.

b. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable oral doses for adults include, for example, about 1 mg/kg/day to about 5 mg/kg/day (usually in combination), depending upon gastrointestinal tolerance; or about 1 mg/kg/day to about 2 mg/kg/day; intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. In some aspects, a dose of about 250 mg/kg/day may be administered as an antineoplastic. Because of gastrointestinal adverse effects, the intravenous route is preferred for loading. During maintenance, a leukocyte count of about 3000/mm³ to 4000/mm³ usually is desired. The drug also sometimes is administered intramuscularly, by infiltration or into body cavities. It is available in dosage forms for injection of about 100 mg, about 200 mg and about 500 mg, and tablets of about 25 mg and about 50 mg.

C. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of about 2.1. Melphalan is available in tablet form for oral administration and has been used to treat multiple myeloma. Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of about 0.2 mg/kg daily for five days as a single course. Courses are repeated about every four to five weeks depending upon hematologic tolerance (Smith and Rutledge, 1975; Young et al., 1978). Alternatively in certain embodiments, the dose of melphalan used could be as low as about 0.05 mg/kg/day or as high as about 3 mg/kg/day or greater.

ii. Ethylenimenes and Methymelamines

An ethylenimene and/or a methylmelamine include, but are not limited to, hexamethylmelamine, used to treat ovary cancer; and thiotepa, which has been used to treat bladder, breast and ovary cancer.

iii. Alkyl Sulfonates

An alkyl sulfonate includes but is not limited to such drugs as busulfan, which has been used to treat chronic granulocytic leukemia.

Busulfan (also known as myleran) is a bifunctional alkylating agent. Busulfan is known chemically as 1,4-butanediol dimethanesulfonate. Busulfan is available in tablet form for oral administration, wherein for example, each scored tablet contains about 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. Busulfan has been shown to be superior to splenic irradiation with respect to survival times and maintenance of hemoglobin levels, and to be equivalent to irradiation at controlling splenomegaly.

iv. Nitrosourea

Nitrosureas, like alkylating agents, inhibit DNA repair proteins. They are used to treat non-Hodgkin's lymphomas, multiple myeloma, malignant melanoma, in addition to brain tumors. A nitrosourea include but is not limited to a carmustine (BCNU), a lomustine (CCNU), a semustine (methyl-CCNU) or a streptozocin. Semustine has been used in such cancers as a primary brain tumor, a stomach or a colon cancer. Stroptozocin has been used to treat diseases such as a malignant pancreatic insulinoma or a malignalnt carcinoid. Streptozocin has beeen used to treat such cancers as a malignant melanoma, Hodgkin's disease and soft tissue sarcomas.

a. Carmustine

Carmustine (sterile carmustine) is one of the nitrosoureas used in the treatment of certain neoplastic diseases. It is 1,3 bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has been used in treating such cancers as a multiple myeloma or a malignant melanoma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

Sterile carmustine is commonly available in 100 mg single dose vials of lyophilized material. The recommended dose of carmustine as a single agent in previously untreated patients is about 150 mg/m² to about 200 mg/m² intravenously every 6 weeks. This may be given as a single dose or divided into daily injections such as about 75 mg/m² to about 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention, for example about 10 mg/m², about 20 mg/m², about 30 mg/m², about 40 mg/m², about 50 mg/m², about 60 mg/M², about 70 mg/m², about 80 mg/M², about 90 mg/m2 to about 100 mg/m².

b. Lomustine

Lomustine is one of the nitrosoureas used in the treatment of certain neoplastic diseases. It is 1-(2-chloro-ethyl)-3-cyclohexyl-l nitrosourea. It is a yellow powder with the empirical formula of C₉H₁₆ClN₃O₂ and a molecular weight of 233.71. Lomustine is soluble in 10% ethanol (about 0.05 mg/mL) and in absolute alcohol (about 70 mg/mL). Lomustine is relatively insoluble in water (less than about 0.05 mg/mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from about 30 mg/m² to 100 mg/m², about half of the radioactivity given was excreted in the form of degradation products within 24 hours. The serum half-life of the metabolites ranges from about 16 hours to about 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. Lomustine has been used to treat such cancers as small-cell lung cancer. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is about 130 mg/m² as a single oral dose every 6 weeks. In individuals with compromised bone marrow function, the dose should be reduced to about 100 mg/m² every 6 weeks. When lomustine is used in combination with other myelosuppressive drugs, the doses should be adjusted accordingly. It is understood that other doses may be used for example, about 20 mg/m², about 30 mg/m², about 40 mg/M², about 50 mg/m², about 60 mg/m², about 70 mg/m², about 80 mg/m², about 90 mg/m², about 100 mg/m² to about 120 mg/m².

C. Triazine

A triazine include but is not limited to such drugs as a dacabazine (DTIC; dimethyltriazenoimidaz olecarboxamide), used in the treatment of such cancers as a malignant melanoma, Hodgkin's disease and a soft-tissue sarcoma.

b. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

i. Folic Acid Analogs

Folic acid analogs include but are not limited to compounds such as methotrexate (amethopterin), which has been used in the treatment of cancers such as acute lymphocytic leukemia, choriocarcinoma, mycosis fungoidesi, breast, head and neck, lung and osteogenic sarcoma.

ii. Pyrimidine Analogs

Pyrimidine analogs include such compounds as cytarabine (cytosine arabinoside), 5-fluorouracil (fluouracil; 5-FU) and floxuridine (fluorode-oxyuridine; FudR). Cytarabine has been used in the treatment of cancers such as acute granulocytic leukemia and acute lymphocytic leukemias. Floxuridine and 5-fluorouracil have been used in the treatment of cancers such as breast, colon, stomach, pancreas, ovary, head and neck, urinary bladder and topical premalignant skin lesions.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

iii. Purine Analogs and Related Inhibitors

Purine analogs and related compounds include, but are not limited to, mercaptopurine (6-mercaptopurine; 6-MP), thioguanine (6-thioguanine; TG) and pentostatin (2-deoxycoformycin). Mercaptopurine has been used in acute lymphocytic, acute granulocytic and chronic granulocytic leukemias. Thrioguanine has been used in the treatment of such cancers as acute granulocytic leukemia, acute lymphocytic leukemia and chronic lymphocytic leukemia. Pentostatin has been used in such cancers as hairy cell leukemias, mycosis fungoides and chronic lymphocytic leukemia.

C. Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, anti-tumor antibiotics, enzymes and biological response modifiers.

i. Mitotic Inhibitors

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

a. Epipodophyllotoxins

Epipodophyllotoxins include such compounds as teniposide and VP16. VP16 is also known as etoposide and is used primarily for treatment of testicular tumors, in combination with bleomycin and cisplatin, and in combination with cisplatin for small-cell carcinoma of the lung. Teniposide and VP16 are also active against cancers such as testis, other lung cancer, Hodgkin's disease, non-Hodgkin's lymphomas, acute granulocytic leukemia, acute nonlymphocytic leukemia, carcinoma of the breast, and Kaposi's sarcoma associated with acquired immunodeficiency syndrome (AIDS).

VP16 is available as a solution (e.g., 20 mg/ml) for intravenous administration and as 50 mg, liquid-filled capsules for oral use. For small-cell carcinoma of the lung, the intravenous dose (in combination therapy) is can be as much as about 100 mg/m² or as little as about 2 mg/m², routinely about 35 mg/m², daily for about 4 days, to about 50 mg/m², daily for about 5 days have also been used. When given orally, the dose should be doubled. Hence the doses for small cell lung carcinoma may be as high as about 200 mg/m² to about 250 mg/m². The intravenous dose for testicular cancer (in combination therapy) is about 50 mg/m² to about 100 mg/m² daily for about 5 days, or about 100 mg/m² on alternate days, for three doses. Cycles of therapy are usually repeated about every 3 to 4 weeks. The drug should be administered slowly (e.g., about 30 minutes to about 60 minutes) as an infusion in order to avoid hypotension and bronchospasm, which are probably due to the solvents used in the formulation.

b. Taxoids

Taxoids are a class of related compounds isolated from the bark of the ash tree, Taxus brevifolia. Taxoids include but are not limited to compounds such as docetaxel and paclitaxel.

Paclitaxel binds to tubulin (at a site distinct from that used by the vinca alkaloids) and promotes the assembly of microtubules. Paclitaxel is being evaluated clinically; it has activity against malignant melanoma and carcinoma of the ovary. In certain aspects, maximal doses are about 30 mg/m² per day for about 5 days or about 210 mg/m² to about 250 mg/m² given once about every 3 weeks.

C. Vinca Alkaloids

Vinca alkaloids are a type of plant alkaloid identified to have pharmaceutical activity. They include such compounds as vinblastine (VLB) and vincristine.

1. Vinblastine

Vinblastine is an example of a plant aklyloid that can be used for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. When the drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in about 7 days to about 10 days. If a moderate level of leukopenia (approximately 3000 cells/mm³) is not attained, the weekly dose may be increased gradually by increments of about 0.05 mg/kg of body weight. In regimens designed to cure testicular cancer, vinblastine is used in doses of about 0.3 mg/kg about every 3 weeks irrespective of blood cell counts or toxicity.

An important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, testis cancer, neuroblastoma, and Letterer-Siwe disease (histiocytosis X), as well as in carcinoma of the breast and choriocarcinoma in women.

Doses of about 0.1 mg/kg to about 0.3 mg/kg can be administered or about 1.5 mg/m² to about 2 mg/m² can also be administered. Alternatively, about 0.1 mg/m², about 0.12 mg/m², about 0.14 mg/m², about 0.15 mg/m², about 0.2 mg/m², about 0.25 mg/m², about 0.5 mg/m², about 1.0 mg/m², about 1.2 mg/m², about 1.4 mg/m², about 1.5 mg/m², about 2.0 mg/m², about 2.5 mg/m², about 5.0 mg/m², about 6 mg/m², about 8 mg/m², about 9 mg/m², about 10 mg/m², to about 20 mg/m², can be given.

2. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is about 0.4 mM.

Vinblastine and vincristine bind to plasma proteins. They are extensively concentrated in platelets and to a lesser extent in leukocytes and erythrocytes. Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than about 3 mg/dl (about 50 mM).

Vincristine sulfate is available as a solution (e.g., 1 mg/ml) for intravenous injection. Vincristine used together with corticosteroids is presently the treatment of choice to induce remissions in childhood leukemia; the optimal dosages for these drugs appear to be vincristine, intravenously, about 2 mg/m² of body-surface area, weekly; and prednisone, orally, about 40 mg/m², daily. Adult patients with Hodgkin's disease or non-Hodgkin's lymphomas usually receive vincristine as a part of a complex protocol. When used in the MOPP regimen, the recommended dose of vincristine is about 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, small cell lung, and carcinomas of the breast, bladder, and the male and female reproductive systems.

Doses of vincristine include about 0.01 mg/kg to about 0.03 mg/kg or about 0.4 mg/m² to about 1.4 mg/m² can be administered or about 1.5 mg/m² to about 2 mg/m² can also be administered. Alternatively, in certain embodiments, about 0.02 mg/m², about 0.05 mg/m², about 0.06 mg/m², about 0.07 mg/m², about 0.08 mg/m², about 0.1 mg/m², about 0.12 mg/m², about 0.14 mg/m², about 0.15 mg/m², about 0.2 mg/m², about 0.25 mg/m² can be given as a constant intravenous infusion.

d. Anti-tumor Antibiotics

Anti-tumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of anti-tumor antibiotics include, but are not limited to, bleomycin, dactinomycin, daunorubicin, doxorubicin (Adriamycin), plicamycin (mithramycin) and idarubicin. Widely used in clinical setting for the treatment of neoplasms these compounds generally are administered through intravenous bolus injections or orally.

1. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,1 0-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of diseases including ovarian, endometrial and breast tumors, bronchogenic oat-cell carcinoma, non-small cell lung carcinoma, stomach, genitourinary, thyroid, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma, soft tissue sarcoma, Ewing's sarcoma, rhabdomyosarcoma and acute lymphocytic leukemia. It is an alternative drug for the treatment of other diseases such as islet cell, cervical, testicular and adrenocortical cancers. It is also an immunosuppressant.

Doxorubicin is absorbed poorly and is preferably administered intravenously. The pharmacokinetics are multicompartmental. Distribution phases have half-lives of 12 minutes and 3.3 hours. The elimination half-life is about 30 hours, with about 40% to about 50% secreted into the bile. Most of the remainder is metabolized in the liver, partly to an active metabolite (doxorubicinol), but a few percent is excreted into the urine. In the presence of liver impairment, the dose should be reduced.

In certain embodiments, appropriate intravenous doses are, adult, about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by about 50% if the serum bilirubin lies between about 1.2 mg/dL and about 3 mg/dL and by about 75% if above about 3 mg/dL. The lifetime total dose should not exceed about 550 mg/m² in patients with normal heart function and about 400 mg/m² in persons having received mediastinal irradiation. In certain embodiments, and alternative dose regiment may comprise about 30 mg/m² on each of 3 consecutive days, repeated about every 4 week. Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

2. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin (daunomycin; rubidomycin) intercalates into DNA, blocks DAN-directed RNA polymerase and inhibits DNA synthesis. It can prevent cell division in doses that do not interfere with nucleic acid synthesis.

In combination with other drugs it is often included in the first-choice chemotherapy of diseases such as, for example, acute granulocytic leukemia, acute myelocytic leukemia in adults (for induction of remission), acute lymphocytic leukemia and the acute phase of chronic myelocytic leukemia. Oral absorption is poor, and it preferably given by other methods (e.g., intravenously). The half-life of distribution is 45 minutes and of elimination, about 19 hours. The half-life of its active metabolite, daunorubicinol, is about 27 hours. Daunorubicin is metabolized mostly in the liver and also secreted into the bile (about 40%). Dosage must be reduced in liver or renal insufficiencies.

Generally, suitable intravenous doses are (base equivalent): adult, younger than 60 years, about 45 mg/m²/day (about 30 mg/m² for patients older than 60 year.) for about 1 day, about 2 days or about 3 days about every 3 weeks or 4 weeks or about 0.8 mg/kg/day for about 3 days, about 4 days, about 5 days to about 6 days about every 3 weeks or about 4 weeks; no more than about 550 mg/m² should be given in a lifetime, except only about 450 mg/m² if there has been chest irradiation; children, about 25 mg/m² once a week unless the age is less than 2 years. or the body surface less than about 0.5 m, in which case the weight-based adult schedule is used. It is available in injectable dosage forms (base equivalent) of about 20 mg (as the base equivalent to about 21.4 mg of the hydrochloride). Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

3. Mitomycin

Mitomycin (also known as mutamycin and/or mitomycin-C) is an antibiotic isolated from the broth of Streptomyces caespitosus which has been shown to have anti-tumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed. Mitomycin has been used in tumors such as stomach, cervix, colon, breast, pancreas, bladder and head and neck.

In humans, mitomycin is rapidly cleared from the serum after intravenous administration. Time required to reduce the serum concentration by about 50% after a 30 mg. bolus injection is 17 minutes. After injection of 30 mg, 20 mg, or 10 mg I.V., the maximal serum concentrations were 2.4 mg/mL, 1.7 mg/mL, and 0.52 mg/mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

4. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is often a component of first-choice combinations for treatment of diseases such as, for example, choriocarcinoma, embryonal rhabdomyosarcoma, testicular tumor, Kaposi's sarcoma and Wilms' tumor. Tumors that fail to respond to systemic treatment sometimes respond to local perfusion. Dactinomycin potentiates radiotherapy. It is a secondary (efferent) immunosuppressive.

In certain specific aspects, actinomycin D is used in combination with agents such as, for example, primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hours. The drug does not pass the blood-brain barrier. Actinomycin D is supplied as a lyophilized powder (0/5 mg in each vial). The usual daily dose is about 10 mg/kg to about 15 mg/kg; this is given intravenously for about 5 days; if no manifestations of toxicity are encountered, additional courses may be given at intervals of about 3 weeks to about 4 weeks. Daily injections of about 100 mg to about 400 mg have been given to children for about 10 days to about 14 days; in other regimens, about 3 mg/kg to about 6 mg/kg, for a total of about 125 mg/kg, and weekly maintenance doses of about 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

5. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of greater than about 35 mL per minute, the serum or plasma terminal elimination half-life of bleomycin is approximately 115 minutes. In patients with a creatinine clearance of less than about 35 mL per minute, the plasma or serum terminal elimination half-life increases exponentially as the creatinine clearance decreases. In humans, about 60% to about 70% of an administered dose is recovered in the urine as active bleomycin. In specific embodiments, bleomycin may be given by the intramuscular, intravenous, or subcutaneous routes. It is freely soluble in water. Because of the possibility of an anaphylactoid reaction, lymphoma patients should be treated with two units or less for the first two doses. If no acute reaction occurs, then the regular dosage schedule may be followed.

In preferred aspects, bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), esophagus, lung and genitourinary tract, Hodgkin's disease, non-Hodgkin's lymphoma, skin, penis, cervix, and vulva. It has also been used in the treatment of lymphomas and testicular carcinoma.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

d. Hormones and Antagonists

Hormonal therapy may also be used in conjunction with the present invention and/or in combination with any other cancer therapy or agent(s). The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

1. Adrenocorticosteroids

Corticosteroid hormones are useful in treating some types of cancer (e.g., non-Hodgkin's lymphoma, acute and chronic lymphocytic leukemias, breast cancer, and multiple myeloma). Though these hormones have been used in the treatment of many non-cancer conditions, they are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

2. Other Hormones and Antagonists

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer. U.S. Pat. No. 4,418,068, incorporated herein by reference, discloses antiestrogenic and antiandrogenic benzothiophenes, such as, for example, 6-hydroxy-2-(4-hydroxyphenyl)-3-[4-(2-piperidinoethoxy)benzoyl]benzo[b]thiophene, and esters, ethers, and salts thereof for the treatment of cancers such as prostate and breast cancer.

e. Miscellaneous Agents

Some chemotherapy agents do not qualify into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they are included within the compositions and methods of the present invention for use in combination therapies.

i. Platinum Coordination Complexes

Platinum coordination complexes include such compounds as carboplatin and cisplatin (cis-DDP). Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes, such as for example, intravenous, subcutaneous, intratumoral or intraperitoneal injection. Cisplatin can be used alone or in combination with other agents, with efficacious doses used in clinical applications of about 15 mg/m² to about 20 mg/m² for 5 days every three weeks for a total of three courses being contemplated in certain embodiments. Doses may be, for example, about 0.50 mg/m², about 1.0 mg/m², about 1.50 mg/m², about 1.75 mg/m², about 2.0 mg/m², about 3.0 mg/m², about 4.0 mg/m², about 5.0 mg/m², to about 10 mg/m².

ii. Other Agents

An anthracenedione such as mitoxantrone has been used for treating acute granulocytic leukemia and breast cancer. A substituted urea such as hydroxyurea has been used in treating chronic granulocytic leukemia, polycythemia vera, essental thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

2. Radiotherapeutic Agents

Radiotherapeutic agents include radiation and waves that induce DNA damage for example, y-irradiation, X-rays, UV-irradiation, microwaves, electronic emissions, radioisotopes, and the like. Therapy may be achieved by irradiating the localized tumor site with the above described forms of radiations. It is most likely that all of these agents effect a broad range of damage DNA, on the precursors of DNA, the replication and repair of DNA, and the assembly and maintenance of chromosomes.

Radiotherapeutic agents and methods of administration, dosages, etc. are well known to those of skill in the art, and may be combined with the invention in light of the disclosures herein. For example, dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

3. Surgery

Approximately 60% of persons with cancer will undergo surgery of some type, which includes, for example, preventative, diagnostic or staging, curative and palliative surgery. Surgery, and in particular a curative surgery, may be used in conjunction with other therapies, such as the present invention and one or more other agents.

Curative surgery includes resection in which all or part of cancerous tissue is physically removed, excised and/or destroyed. It is further contemplated that surgery may remove, excise or destroy superficial cancers, precancers, or incidental amounts of normal tissue. Treatment by surgery includes for example, tumor resection, laser surgery, cryosurgery, electrosurgery, and miscopically controlled surgery (Mohs' surgery). Tumor resection refers to physical removal of at least part of a tumor. Upon excision of part of all of cancerous cells, tissue, or tumor, a cavity may be formed in the body.

Further treatment of the tumor or area of surgery may be accomplished by perfusion, direct injection or local application of the area with an additional anti-cancer agent. Such treatment may be repeated, for example, about every 1, about every 2, about every 3, about every 4, about every 5, about every 6, or about every 7 days, or about every 1, about every 2, about every 3, about every 4, or about every 5 weeks or about every 1, about every 2, about every 3, about every 4, about every 5, about every 6, about every 7, about every 8, about every 9, about every 10, about every 11, or about every 12 months. These treatments may be of varying dosages as well.

4. Immunotherapeutic Agents

An immunotherapeutic agent generally relies on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (e.g., a chemotherapeutic, a radionuclide, a ricin A chain, a cholera toxin, a pertussis toxin, etc.) and serve merely as a targeting agent. Such antibody conjugates are called immunotoxins, and are well known in the art (see U.S. Pat. No. 5,686,072, No. 5,578,706, No. 4,792,447, No. 5,045,451, No. 4,664,911, and No. 5,767,072, each incorporated herein by reference). Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

a. Immune Stimulators

In a specific aspect of immunotherapy is to use an immune stimulating molecule as an agent, or more preferably in conjunction with another agent, such as for example, a cytokines such as for example IL-2, IL-4, IL-12, GM-CSF, tumor necrosis factor; interferons alpha, beta, and gamma; F42K and other cytokine analogs; a chemokine such as for example MIP-1, MIP-1beta, MCP-1, RANTES, IL-8; or a growth factor such as for example FLT3 ligand.

One particular cytokine contemplated for use in the present invention is tumor necrosis factor. Tumor necrosis factor (TNF; Cachectin) is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

Another cytokine specifically contemplate is interferon alpha. Interferon alpha has been used in treatment of hairy cell leukemia, Kaposi's sarcoma, melanoma, carcinoid, renal cell cancer, ovary cancer, bladder cancer, non-Hodgkin's lymphomas, mycosis fungoides, multiple myeloma, and chronic granulocytic leukemia.

b. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. For example, human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers (Bajorin et al., 1988).

C. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

d. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

5. Genetic Therapy Agents

A tumor cell resistance to agents, such as chemotherapeutic and radiotherapeutic agents, represents a major problem in clinical oncology. One goal of current cancer research is to find ways to improve the efficacy of one or more anti-cancer agents by combining such an agent with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that gene therapy could be used similarly in conjunction with the lipid/retinoid compositions of the present invention and/or other agents.

a. Inducers of Cellular Proliferation

In one embodiment of the present invention, it is contemplated that anti-sense mRNA directed to a particular inducer of cellular proliferation is used to prevent expression of the inducer of cellular proliferation. The proteins that induce cellular proliferation further fall into various categories dependent on function. The commonality of all of these proteins is their ability to regulate cellular proliferation.

For example, a form of PDGF, the sis oncogene, is a secreted growth factor. Oncogenes rarely arise from genes encoding growth factors, and at the present, sis is the only known naturally-occurring oncogenic growth factor.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

Other proteins such as Jun, Fos and Myc are proteins that directly exert their effects on nuclear functions as transcription factors.

b. Inhibitors of Cellular Proliferation

In certain embodiment, the restoration of the activity of an inhibitor of cellular proliferation through a genetic construct is contemplated. Tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is pl6. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fms, trk, ret, gsp, hst, abl, E1A, p300, genes involved in angiogenesis (e.g., VEGF, FGF, thrombospondin, BAI-1, GDAIF, or their receptors) and MCC.

C. Regulators of Programmed Cell Death

In certain embodiments, it is contemplated that genetic constructs that stimulate apoptosis will be used to promote the death of diseased or undesired tissue. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri). 6. iNOS Inducers

Nitric oxide signaling has been critically implicated in a variety of functions such as vasodiliation, neurotransmission, host defense and iron metabolism. The diverse actions of NO have been explained by the differential expression and activity of enzymes involved in its synthesis, their regulation, and its chemistry in various biological milieux.

Nitric oxide is produced by a family of enzymes, nitric oxide synthases, that are comprised of three known isoenzymes generally found to be evenly distributed in the cytosol and membrane of cells. These enzymes are: inducible nitric oxide synthase (iNOS); endothelial nitric oxide synthase (eNOS); and neuronal nitric oxide synthase (nNOS). iNOS was originally identified in the mouse marcophage but have since been found to be expressed in numerous cell types such as breast adenocarcinoma. In contrast to its eNOS and nNOs family members, iNOs is not constitutively expressed in cells. Moreover, under standard culture conditions, iNOS has been found to produce micromolar quantities of nitric oxide(NO) in a calcium independent manner. Conversely, NO production by eNOS or nNOS is calcium dependent and in the nanomolar to picomolar range. iNOS has been linked to a number of pathological diseases, particularly cancers such as breast cancer, colon cancer, and cancers of the central nervous system, where an increased level of iNOS activity and/or expression has been observed.

Presence of inducible NOS (iNOS) is dependent on cytokine. It has been demonstrated that the inducible form of NOS is responsive to various agents such as IFNγ (Sun et al, 2000). Furthermore, it has been demonstrated in epithelial cells, that maximal induction of NOS is regulated by cytokines such as IFNγ, TNF-α, and IL-1β (Asano et al., 1994). It has also been demonstrated that the expression of iNOS activity in various cells and tissues, is controlled at the level of transcription by combinations of cytokines (Nathan, 1992; Nussler et al., 1993; Xie et al., 1993; Geller et al., 1993).

Immunosuppressive agents such as cyclosporin can directly modulate cytokine and/or LPS-induced NO production in various cell types in vitro, by interfering with iNOS gene transcription or catalytic activity of the iNOS enzyme (Trajkovic 2001; Attur et al., 2000; Kaibori et al., 1999). Interestingly, some of these drugs exhibited cell-specific pattern of iNOS modulation, thus indirectly indicating distinct requirements for iNOS induction in various cell types. (Trajkovic 2001).

It has also been demonstrated, in breast cancer cells, that expression of iNOS correlated with tumor grade (Tschugguel et al., 1999; Jadeski et al., 1999). Retinoid induced growth inhibition was also observed to correlate with an increase in NO production in breast cancer cells (Martin et al., 2000). Carcinomas with iNOS-positive tumor and stromal cells have a higher apoptotic indices and increased vascularization, suggesting that iNOS contributes to promotion of apoptosis and angiogenesis in breast carcinoma. Furthermore, the expression of iNOS in breast cancer cells has been correlated with the estrogen receptor positivity (Tschugguel et al., 1999; Reveneau et al., 1999).

In accordance with the present invention, it has been determined that the efficacy of 4-HPR as a chemopreventive agent in preventing or reversing breast cancer may be further enhanced in the presence of agents that potentiate its induction of NO production and growth inhibition.

(i) Interferon-gammma (INF-γ)

Interferon gamma (IFN-γ) is induced in T-lymphocytes by foreign antigens for which the T cells have specific receptors. IFN-γ, induces a variety of biological responses such as antiviral, antiproliferative, and immunomodulatory activities in sensitive cells. Immunoregulatory functions induced by IFN-γ such as induction of class I and class II human HLA antigens, activation of macrophages, regulation of Ig class switching, and up-regulation of Fc receptor expression are involved in modulating a variety of other host defense mechanisms. Rather than protecting cells directly, IFN-γ activates surrounding cells by binding to IFN-specific receptors on these cells, thereby activating the production of intracellular effector proteins (Baron et al., 1994). These effector proteins then mediate various immune responses (e.g., antitumor, antiviral, and immunomodulatory). Because of these biological activities, IFN-γ is used as an antitumor agent since its discovery, and has been extensively studied in clinical trials as a therapeutic agent for malignant tumors in general. Its ability to induce iNOS makes it a suitable combination therapy with fenretinide.

(ii) Cyclosporin A

Cyclosporin A belongs to a class of structurally distinctive, cyclic, poly-N-methylated undecapeptides, which include FK506, leflunomide, mycophenolate mofetil, pentoxifylline, and linomide, commonly possessing pharmacological properties. Cyclosporin A has a variety of useful properties such as antibiotic, anti-arthritic and in particular immunosuppressive, anti-inflammatory and/or antiparasitic activities; and may find use in the treatment of conditions such as diabetes, malaria and autoimmune diseases. Cyclosporin A is a highly effective as an immunosuppressant drug, but its use must be carefully managed since the effective dose range is narrow, and excessive dosage can result in serious side effects such as: renal dysfunction, hypertension, cardiovascular cramps, hirsutism, acne, tremor, convulsions, headache, gum hyperplasia, diarrhea, nausea, vomiting, hepatotoxicity, abdominal discomfort, paresthesia, flushing, leukopenia, lymphoma, sinusitis and gynecomastia.

The immunosuppressive action of cyclosporin A is very selective and concentrates on T-cell-dependent mechanisms. Cyclosporin A inhibits the proliferation of T-lymphocytes, which normally respond to a proliferative stimulus (produced, e.g., by antigen or mitogen) with cell replication and cell differentiation but also the release of lymphokines (e.g., various interleukins such as IL-2, IL-3 and IL-4, but also IFN-γ) and in this way start the cellular immune response. Cyclosporin's ability to induce iNOS, possibly via IFN-γ, is exploited in the present invention.

(iii) Herceptin

Herceptin, also referred to as Trastuzumab, is a monoclonal antibody that attaches itself to Her-2,/neu a protein receptor on the surface of cancer cells. By binding to cells, herceptin has been shown to slow the growth and spread of tumors that overexpress the Her-2 protein receptors. Overexpression/amplification of the Her-2/neu has been associated with a worse outcome in patients with breast cancer.

Herceptin has been used extensively in clinical studies where it has slowed the growth of tumors, and in some cases, made tumors disappear altogether. In clinical trials Herceptin has been used in conjunction with chemotherapy such as paclitaxel or anthracyclines/cyclophosphamide (chemotherapies commonly used for the treatment of breast cancer). In clinical trials, result in mild to moderate side effects, the most common adverse events included fever and chills. However, there is an increased incidence of cardiac dysfunction, especially when herceptin is used in conjunction with anthracyclines/cyclophosphamide.

7. Other Biological Agents

It is contemplated that other agents may be used in combination with the present invention to improve the therapeutic efficacy of treatment. These additional agents include, agents that affect the upregulation of cell surface receptors and GAP junctions, cytostatic and differentiation agents, inhibitors of cell adehesion, agents that increase the sensitivity of the hyperproliferative cells to apoptotic inducers, or other biological agents such as for example, hyperthermia.

It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population.

In other embodiments, cytostatic or differentiation agents can be used in combination with the present invention to improve the anti-hyerproliferative efficacy of the treatments.

Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as, for example, the antibody c225, could be used in combination with the present invention to improve the treatment efficacy.

Another form of therapy for use in conjunction with the present invention and/or other agent(s) includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

D. Pharmaceutical Preparations

Pharmaceutical aqueous compositions of the present invention comprise an effective amount of one or more retinoid(s), lipid(s), and/or additional agent(s) dissolved or dispersed in a pharmaceutically acceptable carrier or aqueous medium. The phrases “pharmaceutically or pharmacologically acceptable” refers to molecular entities and compositions that do not produce an adverse, allergic or other untoward reaction when administered to a human. As used herein, “pharmaceutically acceptable carrier” includes any and all solvents, dispersion media, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The actual dosage amount of a composition of the present invention administered to a patient can be determined by physical and physiological factors such as body weight, severity of condition, idiopathy of the patient and on the route of administration. With these considerations in mind, the dosage of a lipid composition for a particular subject and/or course of treatment can readily be determined.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally and/or using aerosol, injection, infusion, continuous infusion, localized perfusion bathing target cells directly or via a catheter and/or lavage. For example, the lipid/retinoid composition and/or additional agent may be formulated for parenteral administration, e.g., formulated for injection via the intravenous, intramuscular or sub-cutaneous routes, though other routes such aerosol administration may be used. The preparation of an aqueous composition that contains at least one lipid/retinoid composition and/or an additional agent as an active ingredient will be known to those of skill in the art in light of the present disclosure, as exemplified by Remington's Pharmaceutical Sciences, 16th Ed. Mack Publishing Company, 1980, incorporated herein by reference. Moreover, for human administration, it will be understood that preparations should meet sterility, pyrogenicity, general safety and purity standards as required by FDA Office of Biological Standards.

Typically, such compositions can be prepared as injectables, either as liquid solutions or suspensions; solid forms suitable for preparing solutions or suspensions upon the addition of a liquid prior to injection can also be prepared; and the preparations can also be emulsified. The compositions will be sterile, be fluid to the extent that easy syringability exists, stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

Although it is most preferred that solutions of lipid/retinoid composition and/or additional agent be prepared in sterile water containing other non-active ingredients, made suitable for injection, solutions of such active ingredients can also be prepared in water suitably mixed with a surfactant, such as hydroxypropylcellulose, if desired. Dispersions can also be prepared in liquid polyethylene glycols, and mixtures thereof and in oils. The carrier can also be a solvent or dispersion medium containing, for example, water, ethanol, polyol (for example, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Upon formulation, solutions will be administered in a manner compatible with the dosage formulation and in such amount as is therapeutically effective. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

It is particularly contemplated that suitable pharmaceutical lipid/retinoid and/or additional agent compositions will generally comprise, but are not limited to, from about 10 to about 100 mg of the desired lipid/retinoid and/or additional agent admixed with an acceptable pharmaceutical diluent or excipient, such as a sterile aqueous solution, to give a final concentration of about 0.25 mg/ml to about 2.5 mg/ml with respect to the conjugate, in, for example, 0.15M NaCl aqueous solution at pH 7.5 to 9.0. The preparations may be stored frozen at −10° C. to −70° C. for at least 1 year.

E. Kits

Any of the compositions described herein may be comprised in a kit. In a non-limiting example, a lipid/retinoid and/or additional agent, may be comprised in a kit. The kits will thus comprise, in suitable container means, a retinoid and a lipid, and/or an additional agent of the present invention. Preferred kits may comprise one or more of the following: at least one retinoid, dimyristoyl phosphatidylcholine, soybean oil, tertiarybutyl alcohol and/or water. A preferred retinoid is N-(4-hydroxyphenyl) retinamide. A preferred agent would be one or more therapeutic or targeting agents.

The kits may comprise a suitably aliquoted lipid/retinoid composition and/or additional agent compositions of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the various components, or premade lipid/retinoid composition and/or additional agent, and any other reagent containers in close confinement for commercial sale. Such containers may include injection or blow-molded plastic containers into which the desired vials are retained.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Preparation of Liposomal 4-HPR

To demonstrate the simplified production of a 4-HPR-liposome composition, 4-HPR was mixed with lipid mixture composed of various ratios of dimyristoyl-phosphatidylcholine (DMPC) and soybean oil in tertiarybutyl alcohol-water mixture (Table 2).

TABLE 2
Incorporation efficiency of 4-HPR in liposomes

| Compo- | 4-HPR:Lipid | 1:17 | 1:10 | | | | 1:5 | 1:15 |
|---|---|---|---|---|---|---|---|---|
| sition of | (w/w) | | | | | | | |
| lip- | DMPC: | 1:0 | 1:0 | 9:1 | | 8:2 | 9:1 | 9:1 |
| osome | soybean oil | | | | | | | |
| | in lipid | | | | | | | |
| | mixture | | | | | | | |
| | Water in | 0 | 1 | 1 | 10 | 1 | 10 | 10 |
| | tertiary | | | | | | | |
| | butyl | | | | | | | |
| | alcohol | | | | | | | |

| | | | | | | | |
|---|---|---|---|---|---|---|---|
| Incorporation | 60.0 | 81.5 | 87.5 | 92.8 | 77.5 | 88.3 | 96.4 |
| efficiency (%) | | | | | | | |

The mixture was frozen in acetone-dry ice bath, and then dried by lyophilzer. It was stored as a powder and resuspended with saline before use.

To determine the incorporation efficiency of 4-HPR into liposomes, liposomal 4-HPR powder was resuspended in saline and any free, unincorporated free 4-HPR was separated by centrifugation at 30,000× g for 1 hour. Liposomal 4-HPR was collected as a pellet and washed three times with saline. 4-HPR concentration was determined by absorbance at 345 nm. To remove the interference by liposomal turbidity, absorbance was measured after liposomal 4-HPR before and after separation was diluted with 1:1 mixture of dimethylsulfoxide and water. The incorporation efficiency was calculated by the following equation:

Incorporation efficiency=OD345 (after separation)/OD345 (before separation)×100%

### EXAMPLE 2

### Anti-Tumor Activity of Liposomal 4-HPR

Anti-tumor activity of liposomal 4-HPR was demonstrated in breast cancer cell lines in terms of growth inhibition (Table 3A and 3B). Breast cancer cell lines were plated in 96 well plates in DMEM/F12 medium supplemented with 5% fetal calf serum and incubated with different concentrations of liposomal 4-HPR and free 4-HPR, respectively. At the end of incubation, the growth of cells was determined by the Celltiter 96 Aqueous nonradioactive cell proliferation assay (Promega Corp., Madison, Wis.) according to the manufacturer's protocol. Growth and viability of cells were determined by MTS assay after treatment with 4-HPR for 3 days (BT-474) and 4 days (MCF7). Growth of treated cells was compared with that of untreated cells cultured under the same conditions, and was expressed as % of untreated cells. Tables 3A and 3B shows the resulting data, which represents at least 3 experiments for each number.

TABLE 3A
Comparison of the inhibitory effect of free- and liposomal 4-HPR
on the growth and viability of MCF-7 cells

| | |
|---|---|
| Concentration of | Growth and viability (%) after treatment with |

| 4-HPR (μM) | Free 4-HPR | Liposomal 4-HPR |
|---|---|---|
| 0.1 | 106.5 ± 7.8 | 112.7 ± 2.4 |
| 0.9 | 77.5 ± 5.8 | 72.9 ± 2.9 |
| 1.5 | 43.8 ± 5.7 | 49.2 ± 3.6 |
| 1.8 | 33.6 ± 2. | 35.0 ± 2.6 |
| 3.6 | 17.8 ± 1.5 | 26.6 ± 3.8 |

TABLE 3B
Comparison of the inhibitory effect of free- and liposomal 4-HPR
on the growth and viability of BT-474 cells

| | |
|---|---|
| Concentration of | Growth and viability (%) after treatment with |

| 4-HPR (μM) | Free 4-HPR | Liposomal 4-HPR |
|---|---|---|
| 0.1 | 100.0 ± 13.4 | 84.9 ± 1 0.4 |
| 0.9 | 108.8 ± 4.1 | 99.3 ± 12.0 |
| 1.5 | 99.4 ± 12.1 | 85.7 ± 8.4 |
| 2.3 | 76.9 ± 5.3 | 82.4 ± 1.6 |
| 3.8 | 52.6 ± 3.7 | 66.0 ± 5.1 |
| 9.0 | 17.1 ± 2.8 | 26.6 ± 2.6 |

### EXAMPLE 3

### Evaluation of Shelf Stability of Liposomal 4-HPR

4-HPR can oxidize and degrade rapidly. Liposomal incorporation of 4-HPR can protect 4-HPR from such degradation. The shelf stability of liposomal 4-HPR with free 4-HPR was compared. Liposomal 4-HPR and free 4-HPR were diluted with 0.9% normal saline at 100 μM concentration, and incubated at room temperature under subdued light. At various time points, absorbance at 345 nm was measured as a parameter of 4-HPR stability. All data are expressed as % of absorbance at time zero, which is considered as 100%. It was demonstrated that liposomal 4-PR has a much longer shelf life and therefore is more stable than free 4-HPR (Table 4). This data indicates that liposomes can alter the pharmacological behavior of 4-HPR.

TABLE 4
Comparison of shelf stability of liposomal and free 4-HPR

| Time at room tempature | | |
|---|---|---|
| (days) | % of free 4-HPR | % of liposomal 4-HPR |
| 0 | 100 | 100 |
| 1 | 85 | 90 |
| 2 | 84 | 96 |
| 3 | 71 | 90 |
| 4 | 61 | 92 |
| 5 | 51 | 97 |
| 7 | 32 | 90 |

### EXAMPLE 4

### Liposomal 4-HPR Induces Nitric Oxide Production and Growth Inhibition in Breast Cancer Cells

Nitric oxide (NO), an important biological second messenger, is known to induce growth inhibition and apoptosis in cells. Nitric oxide (NO) is biosynthesized by isoenzymes called nitric oxide synthases. One such isoenzyme, inducible NOS (iNOS), is present only after cytokine induction, such as interferon-γ (IFN-γ). Furthermore, inducible nitric oxide synthase (iNOS) has been shown to be expressed in breast cancer cells. Here, the ability of liposomal 4-HPR and IFN-γ to induce NO production and growth inhibition in breast cancer positive (Table 5) and breast cancer negative (Table 6) cells is examined by nitrite assays and growth inhibition assays, as known to the skilled artisan.

It is also known to those skilled in the art that nitric oxide (NO) is a biologic mediator derived from the amino acid L-arginine. A family of enzymes, known as nitric oxide synthase (NOS), act upon L-arginine to oxidize one of the guanidino nitrogens to nitric oxide while citrulline is formed from the remainder of the L-arginine molecule. Nitric oxide is a very short-lived free radical and is rapidly oxidized to nitrite and nitrate which are measured as the stable inactive end products of nitric oxide formation.

The data here shows that liposomal 4-HPR in combination with IFN-γ significantly increases NO production and growth inhibition in estrogen receptor (ER)-positive breast cancer cells, but not in ER-negative cells.

TABLE 5
4HPR and interferon-γ (IFN-γ) increased NO production and growth
inhibition in ER-positive MCF-7 cells.

| | | Cell count |
|---|---|---|
| Treatment (5 days) | μM Nitrite /1 × 10⁶ cells | (× 10⁴) |
| Untreated | 7.8 ± 0.1 | 239 |
| 4HPR (1 μM) | 72.6 ± 1.7 | 20 |
| IFN-γ (25 U/ml) | 7.1 ± 3.3 | 254 |
| IFN-γ (50 U/ml) | 6.6 ± 0.5 | 258 |
| 4HPR (1 μM) + IFN-γ (25 U/ml) | 180.9 ± 4.8 | 9 |
| 4HPR (1 μM) + IFN-γ (50 U/ml) | 198.2 ± 2.6 | 9 |

TABLE 6
4HPR and interferon-γ (IFN-γ) increased NO production and growth
inhibition in ER-positive MCF-7 cells.

| | | Cell count |
|---|---|---|
| Treatment (5 days) | μM Nitrite /1 × 10⁶ cells | (× 10⁴) |
| Untreated | 22.6 ± 0.2 | 53 |
| 4HPR (1 μM) | 36.9 ± 5.8 | 34 |
| IFN-γ (25 U/ml) | 28.3 ± 4.1 | 51 |
| IFN-γ (50 U/ml) | 24.4 ± 0.4 | 52 |
| 4HPR (1 μM) + IFN-γ (25 U/ml) | 31.3 ± 1.8 | 34 |
| 4HPR (1 μM) + IFN-γ (50 U/ml) | 75.2 ± 0.1 | 15 |

### EXAMPLE 5

### Cyclosporin A Induces Liposomal 4HPR Production of NO in ER Positive and ER Negative Breast Cancer Cells

Immunosuppressive agents such as cyclosporin A, can directly modulate cytokine and/or LPS-induced NO production in various cell types in vitro, by interfering with iNOS gene transcription or catalytic activity of the iNOS enzyme (Trajkovic 2001; Attur et al., 2000; Kaibori et al., 1999).

Liposomal 4-HPR in combination with cyclosporin A, increased iNOS production of NO in breast cancer cells (Table 3-5). The data shows that in the presence of cylcosporin A, liposomal 4-HPR was able to induce the iNOS production of NO in both ER positive and ER negative breast cancer cells. Furthermore, an increase in the production of NO in these cells was not indicated with other retinoids such as CD437 and ATRA.

TABLE 7
4HPR and cyclosporin A increased NO production in
ER-positive MCF-7 cells.

| Treatment (3 days) | μM Nitrite /1 × 10⁶ cells |
|---|---|
| Untreated | 7.8 |
| cyclosporin A (4 μg/ml) | 11.9 |
| 4HPR (1.0 μM) | 12.5 |
| 4HPR (2.0 μM) | 33.8 |
| 4HPR (1.0 μM) + cyclosporin A (4 μg/ml) | 17.3 |
| 4HPR (2.0 μM) + cyclosporin A (4 μg/ml) | 73.8 |
| CD437 (2 μM) | 71.8 |
| CD437 (2 μM) + cyclosporin A (4 μg/ml) | 54.5 |
| ATRA (1 μM) | 15.1 |
| ATRA (1 μM) + cyclosporin A (4 μg/ml) | 24.0 |

TABLE 8
4HPR and cyclosporin A increased NO production
in ER-negative MDA- MB-231 cells.

| Treatment (3 days) | μM Nitrite /1 × 10⁶ cells |
|---|---|
| Untreated | 19.0 |
| cyclosporin A (4 μg/ml) | 26.3 |
| 4HPR (2.5 μM) | 22.8 |
| 4HPR (6.0 μM) | 26.7 |
| 4HPR (2.5 μM) + cyclosporin A (4 μg/ml) | 35.7 |
| 4HPR (6.0 μM) + cyclosporin A (4 μg/ml) | 315.6 |
| CD437 (2 μM) | 59.3 |
| CD437 (2 μM) + cyclosporin A (4 μg/ml) | 31.1 |
| ATRA (1 μM) | 7.1 |
| ATRA (1 μM) + cyclosporin A (4 μg/ml) | 10.5 |

TABLE 9
Effects of cotreatment with cyclosporin A (CsA) on the IC₅₀ of
retinoids in cellular proliferation assays in various breast
cancer cell lines

| | | IC₅₀ (μM) | | |
|---|---|---|---|---|
| Cell line | Treatment | Without CsA | With CsA | -Fold ↑ |
| MCF-7/WT | 4-HPR | 3.76 | 1.21^{a} | 3.11 |
| | CD 437 | 0.287 | 0.358 | |
| | ATRA | 0.288 | 0.287 | |
| MCF-7/ADR | 4-HPR | 6.66 | 2.70^{b} | 2.47 |
| | CD 437 | 1.58 | 197^{a} (1.19^{b}) | |
| | ATRA | — | — | |
| BT-474 | 4-HPR | 3.61 | 1.99^{a} | 1.81 |
| MDA-MB-453 | 4-HPR | 6.20 | 1.90^{a} | 3.26 |
| MDA-MB-231 | 4-HPR | 7.84 | 1.23^{a} | 6.37 |

^{a}CsA was used at 2 μg/ml of concentration.
^{b}CsA was used at 4 μg/ml of concentration.

### EXAMPLE 7

### Liposomal 4HPR and Herceptin Increased NO Production in ER-Negative Breast Cancer Cells

Since the chemoprevention of ER-negative breast cancer has proven to be a challenge, agents that inhibit or suppress oncogenes known to be associated with ER-negative invasive breast cancer may prove useful in enhancing the efficacy of 4-HPR as a chemopreventive agent. Liposomal 4-HPR has been associated with the suppression of genes linked to ER-negative breast cancer such as the Her2/neu oncogene, and epidermal growth factor receptor (EGFR). Overexpression/amplification of the Her-2/neu oncogene has been associated with poor prognosis for breast cancer. Herceptin, is a biologic agent that targets the human epidermal growth factor receptor-2 (HER2), has been clinically proven in treating breast cancer. The data in Table 10 show that liposomal 4-HPR in combination with herceptin induces iNOS production of NO in ER-negative breast cancer cells. The data further indicate that by blocking or suppressing Her-2/neu expression in ER-negative breast cancer cells, induction of iNOS and it production of NO is increased thereby inducing growth inhibition and apoptosis in these cells.

TABLE 10
4HPR and Herceptin increased NO production in ER-negative breast
cancer cells.

| 4HPR | | | SK-Br-3 μM | |
|---|---|---|---|---|
| (μM) | Herceptin | MDA-MB-453 | Nitrite /1 × 10⁶ cells | BT-474 |
| 0 | − | 19.4 ± 0.9 | 16.6 ± 0.6 | 27.3 ± 0.1 |
| 0 | + | 28.5 ± 1.3 | 35.5 ± 1.2 | 67.7 ± 0.3 |
| 1 | − | 30.9 ± 1.5 | 34.9 ± 1.2 | 45.8 ± 0. |
| 1 | + | 44.9 ± 2.2 | 241.8 ± 9.2 | 115.9 ± 1.0 |
| 2.5 | − | 68.1 ± 3.4 | 75.2 ± 5.1 | 146.9 ± 2.8 |
| 2.5 | + | 102.9 ± 3.4 | 611.7 ± 3.1 | 179.7 ± 3.9 |

The present invention thus identifies that agents that enhance the expression of iNOS, and its subsequent production of NO, are valuable in further enhancing the efficacy of liposomal 4-HPR as a chemopreventive agent in breast cancer

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Asano et al., PNAS 91(21):10089-93,1994.

Attur et al., Inflamm Res. Jan;49(1):20-6, 2000

Bajorin et al., Proc. Annu. Meet. Am. Soc. Clin. Oncol., 7:A967, 1988.

Bakhshi et al., “Cloning the chromosomal breakpoint of t(14;18) human lymphomas: clustering around j(h) on chromosome 14 and near a transcriptional unit on 18,” Cell, 41:899, 1985

Bangham, et al., “Diffusion of univalent Ions across the Lamellae of Swollen Phospholipids” J. Mol. Biol., 13 :23 8-252, 1965.

Baron et al. Antiviral Res. 24: 97-110, 1994.

Blazsek et al., Biomed. Pharmacother. 45:169-177, 1991.

Boehm et al., “Synthesis of high specific activity [³H]-9-cis-retinoic acid and its application for identifying retinoids with unusual binding properties,” J. Med. Chem., 37:408-414, 1994.

Caldas et al., Nat. Genet., 8:27-32,1994.

Canadian Patent No. 1,127,170

Canadian Patent No. 1,127,170

Cassady et al., Abstract Collection of International Symposium on Recent Advance in Chemistry and Molecular Biology of Cancer Research, Beijing, China, 1991, pp. 5-6.

Castaigne et al., Blood 76(9):1704-1709, 1990.

Chambon et al., “The family of retinoid acid nuclear receptors” In Retinoids: 10 Years On. 10-27 J. H. Saurat (ed), Karger/Basel, 1991.

Cheng et al, Cancer Res., 54:5547-5551,1994.

Chomienne et al., Blood 76(9):1710-1717, 1990.

Cleary et al., “Nucleotide Sequence of a t(14;18) Chromosomal Breakpoint in Follicular Lumphoma and Demonstration of a Breakpoint-cluster Region Near a Transcriptionally Active Locus on Chromosome 18,” Proc. Natl Acad. Sci. USA, 82:7439-7443, 1985.

Culver et al., “In vivo gene transfer with retroviral vector-producer cells for treatment of experimental brain tumors,” Science, 256:1550-1552, 1992.

Dawson et al., J. Med. Chem. 24:583-592, 1981.

Dawson, J. Med. Chem. 27:1516-1531, 1984.

Deamer and P. Uster, “Liposome Preparation: Methods and Mechanisms,” in Liposomes (M. Ostro, ed.), Marcel Dekker, Inc., New York (1983), pp. 27-52

Degos, Biomed. Pharmacother. 46:201-209, 1992.

Doepner et al., “Effects of novel retinoids on growth and differentiation of a rhabdomyosarcoma cell line,” Arzneim.-Forsch/Drug Res., 42:1036-1040, 1992.

DRUG CARRIERS IN BIOLOGY AND MEDICINE, G. Gregoriadis ed. (1979) pp. 287-341.

Du et al., Inst. Mater. Med. Chinese Acad. Med. Sci. Beijing 17:331-337, 1982.

Edwards et al., J. Med. Chem. 33:1948-1954, 1990.

EP 0100 839B1

EP 0 263 492 A1

EP 0 263 493 A2

EP 0 274 104 A2

EP 0303 915 B1

EP0 11325B1

EP 0 439 042 A1

EP0552624A1

EP 0 568 898 A1

EP A 010 208

EP A 010 209

EP A 033 095

EP A 09 776

EP 0 273 085

Evain-Brion et al., “Retinoid Acid & Cellular Signal Transduction” In Retinoids: 10 Years On. 46-55; J. H. Saurat(ed) Karger/Basel), 1991.

Formelli et al., “Bioactivities of N-(4-hydroxyphenyl) retinamide and retinol B-glucuronide,” FASEB J, 10:1014-1024, 1996.

Formelli et al., “Five-year administration of fenretinide: pharmacokinetics and effects on plasma retinol concentrations,” J. Clin. Oncol., 11 :2036-2042, 1993.

Fraley and Fornari Kaplan, “Entrapment of a bacterial plasmid in phospholipid vesicles:potential for gene transfer,” Proc. Nat'l. Acad. Sci. USA 76:3348-3352, 1979.

French Patent No. 2,293,193

Gabizon et al., “Effect of liposome composition and other factors on the targeting of liposomes to experimental tumors: biodistribution and imaging studies,” Cancer Research, 50:6371-6378, 1990.

Geller et al., Proc Natl. Acad. Sci. USA, vol. 90, pp. 522-6, 1993.

GB Patent No. 2193095

Ghosh and Bachhawat, “Targeting of liposomes to hepatocytes,” In: Liver Diseases, Targeted Diagnosis and Therapy Using Specific Receptors and Ligands, Wu and Wu (Eds.), Marcel Dekker, New York, pp 87-104, 1991.

Gilliland et al., “Antibody-direct cytotoxide agents: use of monoclonal antibody to direct the action of toxin a chains to colorectal carcinoma cells”, Proc. Natl Acad. Sci. USA, 77:4539-4543, 1980.

Gregoriadis G. and Davis C. “Stability of liposomes in vivo and in vitro is promoted by their cholesterol content and the presence of blood cells,” Biochem Biophys Res Commun., 89(4):1287-1293, 1979.

Goodman and Gilman's “The Pharmacological Basis of Therapeutics,” 8^{th} Ed., 18-32, 43-61, 66-78, 1990.

Hah et al., in vivo 4:153-160, 1990.

Han et al., Chinese J. Cancer Res. 2(3):51-53, 1990.

Harvey et al., J. Org. Chem. 53:3936-3943, 1988.

Harvey et al., J. Org. Chem. 55:6161-6166, 1990.

Hollstein et al., Science, 253:49-53, 1991.

Hussussian et al., Nature Genetics, 15-21, 1994.

Irie and Morton, “Regression of cutaneous metastatic melanoma by intralesional injection with human monoclonal antibody to ganglioside GD2,” Proc. Nat'l Acad. Sci. USA 83:8694-8698, 1986

Irie et al., “Melanoma gangliosides and human monoclonal antibody,” In: Human Tumor Antigens and Specific Tumor Therapy, Metzgar & Mitchell (eds.), Alan R. Liss, Inc., New York, pp. 115-126,1989.

Ito et al., Abstract Collection of International Symposium on Recent Advance in Chemistry and Molecular Biology of Cancer Research, Beijing, China, pp. 68-69, 1991.

Jadeski et al., Am J Pathol. 155(4):1381-90, 1999.

Jing et al., Chinese J. Pharmacol. Toxicol. 6(4):278-280, 1992.

Kamb et al., “A cell cycle regulatory potentially involved in genesis of many tumor types” Science, vol. 264, pp.436-440, 1994.

Kagechika et al., J. Med. Chem. 32:1098-1108, 1989.

Kaibori et al.,. J Hepatol 30(6): 1138-45,1999

Kaneda et al., “Increased expression of DNA cointroduced with nuclear protein in adult rat liver,” Science, 243 :375-378, 1989.

Kaneda et al., “Introduction and expression of the human insulin gene in adult rat liver,” J Biol Chem., 264(21) 12126-12129, 1989.

Kato et al., “Expression of hepatitis B virus surface antigen in adult rat liver. Co-introduction of DNA and nuclear protein by a simplified liposome method,” J Biol Chem., 266(6) 3361-3364, 1991.

Kelloff, G. J., Chemoprevention Branch and Agent Development Committee, Division of Cancer Prevention and Control, National Cancer Institute. Clinical development plan: N-(4-hydroxyphenyl) retinamide. J. Cell Biochem. (Suppl.) 20:176-196, 1994.

Kerr et al., “Apoptosis: A basic biological phenomenon with wide-ranging implication in tissue kinetics,” Br. J. Cancer, 26:239-257, 1972.

Kistler et al., “Teratogenicity or arotinoids (retinoids) in vivo and in vitro,” Arch. Toxicol., 64:616-622, 1990.

Kizaki et al., Seminars in Oncology 19(1):95-105, 1992.

Kliewer et al., (1992) Nature. 355:446-449, 1992.

Lo Coco et al., Blood 77(8):1657-1659, 1991.

Martin et al., Oncolo. Rep. 7(2):219-223, 2000.

Mayer et al., “Human T-lymphotropic virus type iii in high-risk antibody negative homosexual men”, annals of internal medicine, 104:194-96, 1986.

Middleton et al., Biochem. Pharmacol. 43:1167-1179,1992.

Mitchell et al, Proc Natl. Acad Sci. USA, 90, 11693-11697, 1993.

Mitchell et al., “Active-Specific Immunotherapy for Melanoma,” J. Clin. Oncol,. 8(5):856-859, 1990.

Montaldo et al., “N-(4-hydroxyphenyl) retinamide is cytotoxic to melanoma cells in vitro through induction of programmed cell death,” Int. J. Cancer, 81:262-267, 1999.

Moon et al., Cancer Detection and Prevention 16:(1)73-79, 1992.

Morton and Ravindranath, “Current concepts concerning melanoma vaccines,” In Tumor Immunology, Dalgleish (ed.), London: Cambridge University Press, 1-55, 1996.

Morton, Foshag, Hoon, Nizze, Wanek, Chang, Davtyan, Gupta, Elashoff, Irie, “Prolongation of survival in metastatic melanoma after active specific immunotherapy with a new polyvalent melanoma vaccine,” Ann. Surg., 216:463-482, 1992.

Mujoo et al., “Disialoganglioside GD2 on human neuroblastoma cells: target antigen for monoclonal antibody-mediated cytolysis and suppression of tumor growth,” Cancer Res., 47: 1098-1104, 1986.

Nair et al., “BMY 30047: a novel topically active retinoid with low local and systemic toxicity,” J. Pharm. and Exp. Therap., 256:365-370, 1991.

Nair et al., Carcinogenesis 12(1):65-69, 1991.

Nathan C., FASEB J., vol. 6, pp. 3051-3064,1992.

Newton et al., “Structure-activity relationships of retinoids in hamster tracheal organ culture,” Cancer Res. 40(10):3413-3425, 1980.

Nicolau and Sene, “Liposome-mediated DNA transfer in eukaryotic cells: dependence of the transfer efficiency upon the type of liposomes used and the host cell cycle stage,” Biochem. Biophys. Acta, 721:185-190, 1982.

Nicolau et al., “Liposomes as carriers for in vivo gene transfer and expression,” Methods Enzymol., 149:157-176, 1987

Nussler, et al., FASEB J. 6(5),1992.

Nussler, et al., J. Exp. Med. 176: 261-264,1992.

Oikawa et al., “Three novel synthetic retinoids, Re 80, Am 580 and Am 80, all exhibit anti-angiogenic activity in vivo,” Eur. J. Pharm., 249:113-116, 1993.

Pagnan et al., “Apoptosis of human neuroblastoma cells induced by liposome-encapsulated fenretinide,” J. Liposome Res., 8:401-423, 1998.

Pagnan et al.,. “GD2-mediated melanoma cell targeting and cytotoxicity of liposome-entrapped fenretinide,” Int. J. Cancer 81:268-274, 1999.

Perales et al., “Gene transfer in vivo: sustained expression and regulation of genes introduced into the liver by receptor-targeted uptake,” Proc. Natl. Acad. Sci. USA, 91:4086-4090, 1994.

PCT/US 85/01161

PCT/US 89/05040

Pollard et al., “Prevention of primary prostate cancer in Lobund-Wistar rats by N-(4-hydroxyphenyl) retinamide,” Cancer Res. 51:3610-3611, 1991.

Ravindranath and Morton, “Role of gangliosides in active immunotherapy with melanoma vaccine,” Intern. Rev. Immunol., 7: 303-329, 1991.

Remington's Pharmaceutical Sciences, 16th Ed. Mack Publishing Company, 1980

Reveneau et al., Lab Invest., 79:1215-25, 1999.

Rosenberg et al., N. Engl. J. Med., 319: 1676, 1988.

Schulz et al., “Detection of ganglioside GD2 in tumor tissues and sera of neuroblastoma patients,” Cancer Res. 44:5914-5920, 1984.

Serrano et al., Nature, 366:704-707,1993.

Serrano et al., Science, 267:249-252,1995.

Shealy et al., J. Med. Chem. 31:1124-1130, 1988.

Skrede et al., Eur. J. Clin. Investig. 21:574-579, 1991.

Smith and Rutledge, “Chemotherapy in advanced ovarian cancer,” Natl. Cancer Inst. Monogr., 42:141-143, 1975.

Smith et al., J. Clin. Oncol. 10(5):839-864, 1992.

Song et al., Inst. Mater. Med. Chinese Acad. Med. Sci. Beijing 19:576-581, 1984.

Sun et al., J Immunother., 23(2):208-14, 2000.

Swanson et al., J. Pharmacol. Exptl. Therap. 219:632-637, 1981.

Szoka et al., “Procedure for Preparation of Liposomes With Large Internal Aqueous Space . . . ”, Proc. Natl. Acad. Sci., 1978, 75:4194-4198.

Torma et al., “Biologic activities of retinoic acid and 3,4-dehydroretinoic acid in human keratinocytes are similar and correlate with receptor affinities and transactivation properties,” J. Invest. Dermatology, 102:49-54, 1994.

Trajkovic V., Curr Drug Metab 2(3):315-29 2001

Tramposch et al., “The pharmacology of a novel topical retinoid, BMY 30123: comparison with Tretinoin,” J. Pharm. Pharmacol., 44:379-386, 1992.

Tschugguel et al., Breast Cancer Res., Treat., 56: 145-151, 1999.

Tsujimoto et. al, “The t(14;18) chromosome translocation involved in B-cell neoplasms result from mistakes in VDJ joining,” Science, 229:1390, 1985.

Turton et al., “Comparative teratogenicity of nine retinoids in the rat,” Int. J. Exp. Path., 73:551-563, 1992.

U.K. Pat App GB2 190378A

U.S. Pat. Nos. 4,109,880 4,126,693 4,126,698 4,162,282 4,171,318 4,190,594 4,200,647 4,231,944 4,310,505 4,310,546 4,323,581 4,326,055 4,395,575 4,396,553 4,418,068 4,523,042 4,529,600 4,533,254 4,565,863 4,602,034 4,664,911 4,665,098 4,713,465 4,728,575 4,728,578 4,737,323 4,743,400 4,783,533 4,792,447 4,863,969 4,870,219 4,883,613 4,894,480 4,900,478 4,921,706 4,935,560 4,960,908 4,990,703 5,001,276 5,008,291 5,010,107 5,030,764 5,030,767 5,045,451 5,055,622 5,075,333 5,096,713 5,096,924 5,116,954 5,124,083 5,158,773 5,192,544 5,242,909 5,399,757 5,401,511 5,432,260 5,464,870 5,516,792 5,534,261 5,574,177 5,578,706 5,599,953 5,603,872 5,663,377 5,686,072 5,703,130 5,716,982 5,767,072 5,808,111 5,821,254 5,849,718 5,871,727 5,879,703 5,889,155 5,939,277 5,968,940 5,972,911 6,107,090

Wagner et al., Science, 260:1510-1513, 1990.

Weinberg, Science, 254:1138-1146, 1991.

Willhite et al., Tox. and Applied Pharm., 112:144-153, 1992.

WO Patent No. 98/0748

WO Patent No. 99/18933

Wong et al., “Appearance of beta-lactamase activity in animal cells upon liposome-mediated gene transfer,” Gene., 10(2): 87-94, 1980.

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Xie et al., J. Exp. Med., vol. 177, pp. 1779-1784, 1993.

Young et al., N Engl J Med. 7;299(23):1261-1266, 1978.

Zhang et al., Nature. 355:441-446, 1992.

What is claimed is:

1. A method of preparing a pharmaceutical retinoid composition, comprising the step of admixing at least one retinoid or a derivative thereof with dimyristoyl phosphatidylcholine, soybean oil, tertiarybutyl alcohol and water, wherein said admixing produces a mixture comprising liposomes in combination with the at least one retinoid or derivative thereof.

2. The method of claim 1, wherein said retinoid comprises N-(4-hydroxyphenyl) retinamide.

3. The method of claim 1, wherein said derivative thereof comprises at least one derivative of N-(4-hydroxyphenyl) retinamide.

4. The method of claim 1, wherein said method further comprises the step of admixing at least one additional agent with one or more of said retinoid, dimyristoyl phosphatidylcholine, soybean oil, tertiarybutyl alcohol or water.

5. The method of claim 4, wherein said agent further comprises a linking moiety attached to said agent and one or more lipids attached to said linking moeity.

6. The method of claim 5, wherein said one or more lipids are the same as said at least one lipid.

7. The method of claim 4, wherein said agent comprises a diagnostic agent.

8. The method of claim 4, wherein said agent comprises a targeting agent.

9. The method of claim 8, wherein said targeting agent comprises at least one antibody to a tumor.

10. The method of claim 4, wherein said agent comprises an additional therapeutic agent.

11. The method of claim 10, wherein said additional therapeutic agent comprises an anticancer agent.

12. The method of claim 11, wherein the anticancer agent is chemotherapy agent, a radiotherapy agent, an immune therapy agent, a genetic therapy agent, a hormonal therapy agent, a biological agent, an additional retinoid or a retinoid derivative.

13. The method of claim 1, wherein said dimyristoyl phosphatidylcholine and said soybean oil comprise a ratio of greater than 80:20 during admixing.

14. The method of claim 13, wherein said dimyristoyl phosphatidylcholine and said soybean oil comprise a ratio of greater than 85:15 during admixing.

15. The method of claim 14, wherein said dimyristoyl phosphatidyl choline and said soybean oil comprise a ratio of greater than 90:10 during admixing.

16. The method of claim 15, wherein said dimyristoyl phosphatidylcholine and said soybean oil comprise a ratio of greater than 95:5 during admixing.

17. The method of claim 1, wherein the ratio of retinoid or a derivative thereof to lipid is greater than 1:5 during admixing.

18. The method of claim 17, wherein the ratio of retinoid or a derivative thereof to lipid is greater than 1:8 during admixing.

19. The method of claim 18, wherein the ratio of retinoid or a derivative thereof to lipid is greater than 1:10 during admixing.

20. The method of claim 19, wherein the ratio of retinoid or a derivative thereof to lipid is greater than 1:12 during admixing.

21. The method of claim 20, wherein the ratio of retinoid or a derivative thereof to lipid is greater than 1:15 during admixing.

22. The method of claim 21, wherein the ratio of retinoid or a derivative thereof to lipid is greater than 1:17 during admixing.

23. The method of claim 22, wherein the ratio of retinoid or a derivative thereof to lipid is greater than 1:20 during admixing.

24. The method of claim 1, wherein said water comprises greater than 1% during admixing.

25. The method of claim 24, wherein said water comprises greater than 2% during admixing.

26. The method of claim 25, wherein said water comprises greater than 4% during admixing.

27. The method of claim 26, wherein said water comprises greater than 6% during admixing.

28. The method of claim 27, wherein said water comprises greater than 8% during admixing.

29. The method of claim 28, wherein said water comprises greater than 10% during admixing.

30. The method of claim 1, further comprising freezing said mixture.

31. The method of claim 1, further comprising lyophylizing said mixture.

32. The method of claim 31, wherein said lyophylizing produces a powder.

33. The method of claim 31, further comprising resuspending said mixture with an aqueous solvent.

34. The method of claim 33, wherein said aqueous solvent comprises a pharmaceutically acceptable saline solution.

35. The method of claim 1, wherein greater than 90% of said retinoid or a derivative thereof is incorporated into said liposomes.

36. The method of claim 35, wherein greater than 91% of said retinoid or a derivative thereof is incorporated into said liposomes.

37. The method of claim 36, wherein greater than 92% of said retinoid or a derivative thereof is incorporated into said liposomes.

38. The method of claim 37, wherein greater than 93% of said retinoid or a derivative thereof is incorporated into said liposomes.

39. The method of claim 38, wherein greater than 94% of said retinoid or a derivative thereof is incorporated into said liposomes.

40. The method of claim 39, wherein greater than 95% of said retinoid or a derivative thereof is incorporated into said liposomes.

41. The method of claim 40, wherein greater than 96% of said retinoid or a derivative thereof is incorporated into said liposomes.

42. A method of preparing a pharmaceutical retinoid composition, comprising the step of admixing N-(4-hydroxyphenyl) retinamide or a derivative thereof with dimyristoyl phosphatidylcholine, soybean oil, tertiarybutyl alcohol and water, wherein said admixing produces a mixture comprising liposomes in combination with the N-(4-hydroxyphenyl) retinamide or a derivative thereof.

43. A pharmaceutical retinoid composition comprising N-(4-hydroxyphenyl) retinamide, or a derivative thereof, encapsulated in a lipid material, wherein said lipid material comprises dimyristoyl phosphatidylcholine and soybean oil.

44. The pharmaceutical composition of claim 43, wherein said dimyristoyl phosphatidylcholine and soybean oil comprise a ratio of greater than 80:20.

45. The pharmaceutical composition of claim 43, wherein said composition further comprises at least one additional agent.

46. The pharmaceutical composition of claim 45, wherein said agent further comprises a linking moiety attached to said agent and one or more lipids of said lipid material attached to said linking moeity.

47. The pharmaceutical composition of claim 45, wherein said agent comprises a targeting agent.

48. The pharmaceutical composition of claim 47, wherein said targeting agent comprises at least one antibody to a tumor.

49. The pharmaceutical composition of claim 45, wherein said agent comprises an additional therapeutic agent.

50. The pharmaceutical composition of claim 49, wherein said additional therapeutic agent comprises an anticancer agent.

51. The method of claim 50, wherein the anticancer agent is chemotherapy agent, a radiotherapy agent, an immune therapy agent, a genetic therapy agent, a hormonal therapy agent, a biological agent, an additional retinoid or a retinoid derivative.

52. The pharmaceutical composition of claim 43, wherein said composition is comprised as a lyophylized material.

53. The pharmaceutical composition of claim 43, wherein said composition is comprised in a pharmaceutically acceptable aqueous medium.

54. A method of treating a subject having cancer, comprising administering to said individual a therapeutically effective amount of a composition comprising N-(4-hydroxyphenyl) retamide, or a derivative thereof, encapsulated in a lipid material, wherein said lipid material comprises dimyristoyl phosphatidylcholine and soybean oil.

55. The method of claim 54, wherein said dimyristoyl phosphatidylcholine and soybean oil comprise a ratio of greater than 80:20.

56. The method of claim 54, wherein said composition is comprised in a pharmaceutically acceptable aqueous medium.

57. The method of claim 54, wherein said method further comprises administering at least one additional therapeutic agent to said individual.

58. The method of claim 58, wherein said agent is comprised in said composition.

59. The method of claim 57, wherein said additional therapeutic agent comprises at least one anticancer agent.

60. The method of claim 59, wherein the anticancer agent is chemotherapy agent, a radiotherapy agent, an immune therapy agent, a genetic therapy agent, a hormonal therapy agent, a biological agent, an additional retinoid or a retinoid derivative.

61. A method for increasing growth inhibitory effects of fenretinide on a cell comprising providing to a cell, in combination with fenretinide, one or more agents that increases the level of nitric oxide (NO) in said cell.

62. The method of claim 61, wherein said cell is a tumor cell.

63. The method of claim 62, wherein said tumor cell is a breast cancer cell.

64. The method of claim 63, wherein the breast cancer cell is an estrogen receptor (ER)-positive cell.

65. The method of claim 63, wherein the breast cancer cell is an estrogen receptor (ER)-negative cell.

66. The method of claim 61, wherein fenretinide is provided before the one or more agents.

67. The method of claim 61, wherein fenretinide is provided at the same time as the one or more agents.

68. The method of claim 61, wherein fenretinide is provided after the one or more agents.

69. The method of claim 61, wherein fenretinide is provided more than once.

70. The method of claim 69, wherein fenretinide is provided daily for three months with monthly three-day interruptions.

71. The method of claim 61, wherein said agent is provided more than once.

72. The method of claim 61, wherein said agent is a nucleic acid.

73. The method of claim 72, wherein said nucleic acid is an expression construct encoding iNOS, interferon-y or herceptin.

74. The method of claim 61, wherein said agent is a protein.

75. The method of claim 74, wherein said protein is iNOS, interferon-y or herceptin.

76. The method of claim 61, wherein said agent is a chemopharmaceutical.

77. The method of claim 76, wherein said agent is cyclosporin A.

78. The method of claim 62, wherein said cell tumor cell is a patient.

79. The method of claim 78, wherein said cell tumor cell is part of a tumor mass in said patient.

80. The method claim 78, wherein providing comprises direct administration to said tumor cell.

81. The method of claim 61, further comprising providing to said cell an additional anti-cancer therapy.

82. The method of claim 81, wherein said additional anti-cancer therapy is radiation.

83. The method of claim 81, wherein said additional anti-cancer therapy is a distinct chemotherapy.

84. The method of claim 81, wherein said additional anti-cancer therapy is a distinct gene therapy.

85. The method of claim 81, wherein said additional anti-cancer therapy is immunotherapy.

86. The method of claim 81, wherein said additional anti-cancer therapy is hormonal therapy.

87. The method of claim 61, wherein fenretinide is provided in an amount sufficient to achieve an intracellular concentration of 0.1 μm.

88. The method of claim 61, wherein fenretinide is provided in an amount sufficient to achieve an intracellular concentration of 0.5 μm.

89. The method of claim 61, wherein fenretinide is provided in an amount sufficient to achieve an intracellular concentration of 1.0 μm.

90. The method of claim 61, wherein said cell is killed.

91. A method for treating cancer in a subject comprising providing to said subject, in combination, fenretinide and one or more agents that increases the level of nitric oxide (NO) in cancer cells in said subject.

92. The method of claim 91, wherein said cancer is a breast cancer.

93. The method of claim 92, wherein cells of said breast cancer are estrogen receptor (ER)-positive.

94. The method of claim 92, wherein cells of said breast cancer are estrogen receptor (ER)-negative.

95. The method of claim 91, wherein fenretinide is provided before the one or more agents.

96. The method of claim 91, wherein fenretinide is provided at the same time as the one or more agents.

97. The method of claim 91, wherein fenretinide is provided after the one or more agents.

98. The method of claim 91, wherein fenretinide is provided more than once.

99. The method of claim 98, wherein fenretinide is provided daily for three months with monthly three-day interruptions.

100. The method of claim 91, wherein said agent is provided more than once.

101. The method of claim 91, wherein said agent is a nucleic acid.

102. The method of claim 101, wherein said nucleic acid is an expression construct encoding iNOS, interferon-y or herceptin.

103. The method of claim 91, wherein said agent is a protein.

104. The method of claim 103, wherein said protein is iNOS, interferon-γ or herceptin.

105. The method of claim 91, wherein said agent is a chemopharmaceutical.

106. The method of claim 105, wherein said agent is cyclosporin A.

107. The method claim 91, wherein providing comprises direct administration to said tumor cell.

108. The method of claim 91, further comprising providing to said cell an additional anti-cancer therapy.

109 The method of claim 108, wherein said additional anti-cancer therapy is radiation.

110. The method of claim 108, wherein said additional anti-cancer therapy is a distinct chemotherapy.

111. The method of claim 108, wherein said additional anti-cancer therapy is a distinct gene therapy.

112. The method of claim 108, wherein said additional anti-cancer therapy is immunotherapy.

113. The method of claim 108, wherein said additional anti-cancer therapy is hormonal therapy.

114. The method of claim 91, wherein fenretinide is provided in an amount sufficient to achieve an intracellular concentration in cancer cells of 0.1 μm.

115. The method of claim 91, wherein fenretinide is provided in an amount sufficient to achieve an intracellular concentration in cancer cells of 0.5 μm.

116. The method of claim 91, wherein fenretinide is provided in an amount sufficient to achieve an intracellular concentration in cancer cells of 1.0 μm.

117. The method of claim 91, wherein fenretinide is provided at 10 mg/day.

118. The method of claim 91, wherein fenretinide is provided at 100 mg/day.

119. The method of claim 91, wherein fenretinide is provided at 200 mg.day.

120. A composition of matter comprising fenretinide and an agent that increases the level of nitric oxide (NO) in a cell.

121. The composition of claim 120, wherein said agent is a nucleic acid.

122. The composition of claim 121, wherein said nucleic acid is an expression construct encoding iNOS, interferon-y or herceptin.

123. The composition of claim 120, wherein said agent is a protein.

124. The composition of claim 123, wherein said protein is iNOS, interferon-γ or herceptin.

125. The composition of claim 129, wherein said agent is a chemopharmaceutical.

126. The composition of claim 125, wherein said agent is cyclosporin A.

127. The composition of claim 120, wherein said composition is encapsulated in a lipid material.

128. The composition of claim 127, wherein said liposome comprises dimyristoyl phosphatidylcholine and soybean oil.

129. A kit comprising fenretinide and at an agent that increases the level of nitric oxide (NO) in a cell.

130. A method for inhibiting metastasis in a subject having cancer comprising providing to said subject, in combination, fenretinide and one or more agents that increases the level of nitric oxide (NO) in cancer cells in said subject.
